(19) United States
(12) Patent Application Publication
(10) Pub. No.: US 2023/0113844 A1
(43) Pub. Date: Apr. 13, 2023

(54) SULFATE FREE CLEAR PERSONAL CLEANSING COMPOSITION COMPRISING LOW INORGANIC SALT AND HYDROXAMIC ACID OR HYDROXAMIC ACID DERIVATIVES

(71) Applicant: The Procter & Gamble Company, Cincinnati, OH (US)

(72) Inventors: Brooke Michele Cochran, Cincinnati, OH (US); Eric Scott Johnson, Hamilton, OH (US); Karen Michelle Nally, Milford, OH (US); Sean Michael Renock, Loveland, OH (US)

(21) Appl. No.: 17/961,148

(22) Filed: Oct. 6, 2022

Related U.S. Application Data
(60) Provisional application No. 63/253,377, filed on Oct. 7, 2021.

(51) Int. Cl. A61K 8/46; A61K 8/19; A61K 8/73; A61K 8/44; A61Q 5/02; A61Q 5/12

(52) U.S. Cl. CPC A61K 8/466; A61K 8/19; A61K 8/737; A61K 8/731; A61K 8/442; A61Q 5/02; A61Q 5/12

(57) ABSTRACT

A clear cleansing composition directed to from about 3 wt % to about 35 wt % of an anionic surfactant; from about 5 wt % to about 15% of an amphoteric surfactant; from about 0.01 wt % to about 2 wt % of a cationic polymer; from about 0 wt % to about 1.0 wt % of inorganic salts; from about 0.01% to about 10% of a hydroxamic acid or hydroxamic acid derivative; an aqueous carrier, wherein the composition is substantially free of sulfate based surfactant and wherein the composition has a % T value of greater than about 70.

### FIELD OF THE INVENTION

The present disclosure generally relates to stable clear personal cleansing compositions which are formulated with anionic surfactants substantially free from sulfates, amphoteric or amphoteric surfactants, cationic deposition polymers, a low level of inorganic salt and a hydroxamic acid or hydroxamic acid derivatives.

### BACKGROUND OF THE INVENTION

Most commercial cleansing compositions, such as shampoo compositions, comprise sulfate-based surfactant systems because of their effectiveness in generating high lather volume and good lather stability and cleaning. However, some consumers may prefer a shampoo composition that is substantially free of sulfate-based surfactant systems. In addition, sulfate free shampoos users prefer high conditioning shampoos because higher conditioning shampoos feel less stripping to the hair. Conditioning shampoos based on sulfate based surfactant systems typically contain cationic conditioning polymers to form coacervate with the sulfate based surfactant system during use. However, it can be difficult to use non-sulfate based surfactants in liquid shampoos because it can be difficult to formulate a composition that has acceptable lather volume, cleansing, conditioning benefit, and stability. One common problem is that using cationic conditioning polymers in products that are substantially free of sulfate containing surfactants can result in instability. In particular, many shampoo compositions that contain non-sulfate based surfactants have a relatively high salt content that can cause an in situ coacervate phase to form in the composition prior to use (rather than the desired formation during use). This in situ coacervate is observed by the consumer as a cloudy product or a product with a precipitated layer, which is not consumer preferred. Presence of coacervate in the cleaning compositions can lead to separation upon storage, causing inconsistent performance in use. It has been found that in situ coacervate can be prevented from forming prior to use by decreasing the salt concentration of the shampoo composition. However, this can cause the viscosity of the shampoo composition to become too low, making it difficult to hold in a user's hand and apply to the hair and scalp. In these low salt compositions, the viscosity can be increased by decreasing the pH. However, many sulfate-free surfactant systems can hydrolyze at low pH resulting in viscosity and performance changes over time and will eventually lead to phase separation.

Therefore, there is a need for a stable shampoo product with sufficient viscosity as made, consistent viscosity over time and superior product performance that contains one or more non-sulfated anionic surfactants, amphoteric surfactants and cationic polymers without forming the in situ coacervate phase in the product prior to dilution with water.

It has been surprisingly found that stable products containing one or more non-sulfated anionic surfactants, amphoteric surfactants and cationic polymers that exhibit good viscosity as made, consistent viscosity over time and good conditioning can be achieved with a combination of low inorganic salt concentration and a hydroxamic acid or hydroxamic acid derivative.

### SUMMARY OF THE INVENTION

A clear cleansing composition comprising from about 3 wt % to about 35 wt % of an anionic surfactant; from about 5 wt % to about 15% of an amphoteric surfactant; from about 0.01 wt % to about 2 wt % of a cationic polymer; from about 0 wt % to about 1.0 wt % of inorganic salts; from about 0.01% to about 10% of a hydroxamic acid or hydroxamic acid derivative; an aqueous carrier, wherein the composition is substantially free of sulfate based surfactant and wherein the composition has a % T value of greater than about 70.

### DETAILED DESCRIPTION OF THE INVENTION

While the specification concludes with claims particularly pointing out and distinctly claiming the invention, it is believed that the present disclosure will be better understood from the following description.

As used herein, the term “fluid” includes liquids and gels.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, “molecular weight” or “M.Wt.” refers to the weight average molecular weight unless otherwise stated. Molecular weight is measured using industry standard method, gel permeation chromatography (“GPC”). The molecular weight has units of grams/mol.

As used herein, “cleansing composition” includes personal cleansing products such as shampoos, conditioners, conditioning shampoos, shower gels, liquid hand cleansers, facial cleansers, and other surfactant-based liquid compositions.

As used herein, the terms “include,” “includes,” and “including,” are meant to be non-limiting and are understood to mean “comprise,” “comprises,” and “comprising,” respectively.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Cleansing Compositions

Typically, inorganic salt is added to sulfated surfactant based cleansing formulations to thicken the product. It has been found that adding inorganic salt to the formulas that are substantially free of sulfate containing surfactants and/or using high inorganic salt containing sulfate free surfactants in the presence of cationic conditioning polymer can cause product instability due to formation of an undesired gel-like phase known as coacervate in the composition (referred to herein as “in situ coacervate” or an “in situ coacervate phase”, which is a coacervate that forms in the composition, prior to dilution, as opposed to when it is diluted with water when a user washes their hair). By maintaining low inorganic salt concentration in formulas (from about 0 to about 1 wt %) the instability issue in sulfate free formulations comprising anionic surfactant and cationic polymer is resolved. The inorganic salt can include sodium chloride, potassium chloride, sodium sulfate, ammonium chloride, sodium bromide, and combinations thereof. The solution is to avoid or minimize adding extra inorganic salt to the formula and/or by using low inorganic salt containing raw materials. For example, commercially available sulfate free surfactants such as disodium cocoyl glutamate typically comes with high levels of inorganic salt such as 5% or higher. Amphoteric surfactant such as betaines or sultaines also typically come with high levels of inorganic salt such as sodium chloride. Use of these high salt containing raw materials in sulfate-free surfactant based cleaning formulations in excess of about 1% total sodium chloride in the formulation can cause formation of undesired in situ coacervate in the product. If the inorganic salt level is lowered in the surfactant raw materials so that total salt in the composition is less than about 1% or lower, a stable 1-phase product can be formulated. Whereas, if the regular material with high inorganic salt is used, the product is cloudy, 2-phase, and unstable. The solution described herein prevents the undesired in situ coacervate formation in product while on the shelf (before use), and yet forms coacervate when needed, during use after dilution, to deliver consumer desired wet conditioning.

The formation of coacervate upon dilution of the cleansing composition with water, rather than while in the bottle on the shelf, is important to improving wet conditioning and deposition of various conditioning actives, especially those that have small droplet sizes (i.e., ≤2 microns). In order to form coacervate at the right time (upon dilution during use) cleansing compositions comprising anionic surfactants substantially free of sulfates, amphoteric surfactants and cationic polymers should maintain an inorganic salt level of less than 1%.

Compositions containing inorganic salt level of less than 1% generally have a viscosity that is too low, which is not consumer preferred because it is difficult to use the product. In these low salt compositions, the viscosity can be increased by decreasing the pH. However, many sulfate-free surfactant systems can hydrolyze at low pH resulting in viscosity and performance changes over time and will eventually lead to phase separation. It has been surprisingly found that a stable shampoo composition with an acceptable and consistent viscosity as made and over time and acceptable product performance could be made with an inorganic salt level of less than 1% if a hydroxamic acid or hydroxamic acid derivative is also used in the composition.

Another benefit of the higher viscosity shampoo composition is that a broader range of formulas with acceptable viscosity can be designed because other formula ingredients are not required to build viscosity. For example, viscosity modifiers, other than an inorganic salt, may not be needed. The composition may be free of or substantially free of viscosity modifiers, other than inorganic salt (e.g., sodium chloride, potassium chloride, sodium sulfate, ammonium chloride, sodium bromide, and combinations thereof), which can include carbomers, cross-linked acrylates, hydrophobically modified associative polymers and cellulose, as described in US Pub. Nos. 2019/0105246 and 2019/010524, incorporated by reference. This can make the shampoo easier to distribute across a user's hair and scalp.

It may be consumer desirable to have a shampoo composition with a minimal level of ingredients. The shampoo composition can be formulated without polymeric thickeners or suspending agents such as carbomer, EGDS or thixcin. The shampoo composition may be comprised of 11 or fewer ingredients, 10 or fewer ingredients, 9 or fewer ingredients, 8 or fewer ingredients, 7 or fewer ingredients, 6 or fewer ingredients. The minimal ingredient formula can include water, anionic surfactant, amphoteric surfactant, cationic polymer, inorganic salt, and perfume. It is understood that perfumes can be formed from one or more materials. In some examples, the composition can be free of or substantially free of fragrance. In another example, the composition can be free of or substantially free of PEG.

The cleansing composition may have has less than 1 wt % of inorganic salt; may have from about 0 wt % to about 0.9 wt % of inorganic salt, may have from about 0 wt % to about 0.8 wt % of inorganic salt, may have from about 0 wt % to about 0.5 wt %, and may have from about 0 wt % to about 0.2 wt %. The shampoo composition can contain no viscosity modifier other than one or more inorganic salts.

The pH can be from about 4 to about 8, alternatively from about 4.5 to about 7.5, alternatively from about 5 to about 7, alternatively from about 5.5 to about 6.5, alternatively from about. 5.5 to about 6, and alternatively from about 6 to about 6.5, as determined by the pH Test Method, described herein. The pH may be greater than about 5.0; may be greater than 5.25; may be greater than 5.5; may be greater than 5.75; may be greater than 6.0.

The cleansing composition is clear prior to dilution with water. The term “clear” or “transparent” as used herein, means that the compositions have a percent transparency (%T) of at least about 70% transmittance at 600 nm. The %T may be at 600 nm from about 70% to about 100%, from about 80% to about 100%, from about 90% to about 100%. In the present invention, the percent transparency (%T) may be at least about 80% transmittance at 600 nm; percent transparency (%T) may be at least about 90% transmittance at 600 nm.

### A. Surfactant

The cleansing compositions described herein can include one or more surfactants in the surfactant system. The one or more surfactants can be substantially free of sulfate-based surfactants. As can be appreciated, surfactants provide a cleaning benefit to soiled articles such as hair, skin, and hair follicles by facilitating the removal of oil and other soils. Surfactants generally facilitate such cleaning due to their amphiphilic nature which allows for the surfactants to break up, and form micelles around, oil and other soils which can then be rinsed out, thereby removing them from the soiled article. Suitable surfactants for a cleansing composition can include anionic moieties to allow for the formation of a coacervate with a cationic polymer. The surfactant can be selected from anionic surfactants, amphoteric surfactants, zwitterionic surfactants, non-ionic surfactants, and combinations thereof.

Cleansing compositions typically employ sulfate-based surfactant systems (such as, but not limited to, sodium lauryl sulfate) because of their effectiveness in lather production, stability, clarity and cleansing. The cleansing compositions described herein are substantially free of sulfate-based surfactants. “Substantially free” of sulfate based surfactants as used herein means from about 0 wt % to about 3 wt %, alternatively from about 0 wt % to about 2 wt %, alternatively from about 0 wt % to about 1 wt %, alternatively from about 0 wt % to about 0.5 wt %, alternatively from about 0 wt % to about 0.25 wt %, alternatively from about 0 wt % to about 0.1 wt %, alternatively from about 0 wt % to about 0.05 wt %, alternatively from about 0 wt % to about 0.01 wt %, alternatively from about 0 wt % to about 0.001 wt %, and/or alternatively free of sulfates. As used herein, “free of” means 0 wt %.

Additionally, the surfactant systems described herein have from about 0 wt % to about 1 wt % of inorganic salts.

Additionally, the surfactants can be added to the composition as a solution, instead of the neat material and the solution can include inorganic salts that can be added to the formula. The surfactant formula can have inorganic salt that can be from about 0% to about 2% of inorganic salts of the final composition, alternatively from about 0.1% to about 1.5%, and alternatively from about 0.2% to about 1%.

Suitable surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

The concentration of the surfactant in the composition should be sufficient to provide the desired cleaning and lather performance. The cleansing composition can comprise a total surfactant level of from about 6% to about 50%, from about 5% to about 35%, a total surfactant level of from about 10% to about 50%, by weight, from about 15% to about 45%, by weight, from about 20% to about 40%, by weight, from about 22% to about 35%, and/or from about 25% to about 30%.

The surfactant system can include one or more amino acid based anionic surfactants. Non-limiting examples of amino acid based anionic surfactants can include sodium, ammonium or potassium salts of acyl glycinates; sodium, ammonium or potassium salts of acyl sarcosinates; sodium, ammonium or potassium salts of acyl glutamates; sodium, ammonium or potassium salts of acyl alaninates and combinations thereof.

The amino acid based anionic surfactant can be a glutamate, for instance an acyl glutamate. The composition can comprise an acyl glutamate level from about 2% to about 22%, by weight, from about 3% to about 19%, by weight, 4% to about 17%, by weight, and/or from about 5% to about 15%, by weight.

Non-limiting examples of acyl glutamates can be selected from the group consisting of sodium cocoyl glutamate, disodium cocoyl glutamate, ammonium cocoyl glutamate, diammonium cocoyl glutamate, sodium lauroyl glutamate, disodium lauroyl glutamate, sodium cocoyl hydrolyzed wheat protein glutamate, disodium cocoyl hydrolyzed wheat protein glutamate, potassium cocoyl glutamate, dipotassium cocoyl glutamate, potassium lauroyl glutamate, dipotassium lauroyl glutamate, potassium cocoyl hydrolyzed wheat protein glutamate, dipotassium cocoyl hydrolyzed wheat protein glutamate, sodium capryloyl glutamate, disodium capryloyl glutamate, potassium capryloyl glutamate, dipotassium capryloyl glutamate, sodium undecylenoyl glutamate, disodium undecylenoyl glutamate, potassium undecylenoyl glutamate, dipotassium undecylenoyl glutamate, disodium hydrogenated tallow glutamate, sodium stearoyl glutamate, disodium stearoyl glutamate, potassium stearoyl glutamate, dipotassium stearoyl glutamate, sodium myristoyl glutamate, disodium myristoyl glutamate, potassium myristoyl glutamate, dipotassium myristoyl glutamate, sodium cocoyl/hydrogenated tallow glutamate, sodium cocoyl/palmoyl/sunfloweroyl glutamate, sodium hydrogenated tallowoyl Glutamate, sodium olivoyl glutamate, disodium olivoyl glutamate, sodium palmoyl glutamate, disodium palmoyl Glutamate, TEA-cocoyl glutamate, TEA-hydrogenated tallowoyl glutamate, TEA-lauroyl glutamate, and mixtures thereof.

The amino acid based anionic surfactant can be an alaninate, for instance an acyl alaninate. Non-limiting example of acyl alaninates can include sodium cocoyl alaninate, sodium lauroyl alaninate, sodium N-dodecanoyl-1-alaninate and combination thereof. The composition can comprise an acyl alaninate level from about 2% to about 20%, by weight, from about 7% to about 15%, by weight, and/or from about 8% to about 12%, by weight.

The amino acid based anionic surfactant can be a sarcosinate, for instance an acyl sarcosinate. Non-limiting examples of sarcosinates can be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

The amino acid based anionic surfactant can be a glycinate for instance an acyl glycinate. Non-limiting example of acyl glycinates can include sodium cocoyl glycinate, sodium lauroyl glycinate and combination thereof.

The composition can contain additional anionic surfactants selected from the group consisting of sulfosuccinates, isethionates, sulfonates, sulfoacetates, glucose carboxylates, alkyl ether carboxylates, acyl taurates, and mixture thereof.

Non-limiting examples of sulfosuccinate surfactants can include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid, dioctyl esters of sodium sulfosuccinic acid, and combinations thereof. The composition can comprise a sulfosuccinate level from about 2% to about 22%, by weight, from about 3% to about 19%, by weight, 4% to about 17%, by weight, and/or from about 5% to about 15%, by weight.

Suitable isethionate surfactants can include the reaction product of fatty acids esterified with isethionic acid and neutralized with sodium hydroxide. Suitable fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, and mixtures thereof.

Non-limiting examples of sulfonates can include alpha olefin sulfonates, linear alkylbenzene sulfonates, sodium laurylglucosides hydroxypropylsulfonate and combination thereof.

Non-limiting examples of sulfoacetates can include sodium lauryl sulfoacetate, ammonium lauryl sulfoacetate and combination thereof.

Non-limiting example of glucose carboxylates can include sodium lauryl glucoside carboxylate, sodium cocoyl glucoside carboxylate and combinations thereof.

Non-limiting example of alkyl ether carboxylate can include sodium laureth-4 carboxylate, laureth-5 carboxylate, laureth-13 carboxylate, sodium C12-13 pareth-8 carboxylate, sodium C12-15 pareth-8 carboxylate and combination thereof.

Non-limiting example of acyl taurates can include sodium methyl cocoyl taurate, sodium methyl lauroyl taurate, sodium caproyl methyltaurate, sodium methyl oleoyl taurate and combination thereof.

The surfactant system may further comprise one or more amphoteric surfactants and the amphoteric surfactant can be selected from the group consisting of betaines, sultaines, hydroxysultanes, amphohydroxypropyl sulfonates, alkyl amphoactates, alkyl amphodiacetates and combination thereof.

Examples of betaine amphoteric surfactants can include coco dimethyl carboxymethyl betaine, cocoamidopropyl betaine (CAPB), cocobetaine, lauryl amidopropyl betaine (LAPB), coco-betaine, cetyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and mixtures thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

Non-limiting example of alkylamphoacetates can include sodium cocoyl amphoacetate, sodium lauroyl amphoacetate and combination thereof.

The amphoteric surfactant can comprise cocamidopropyl betaine (CAPB), lauramidopropyl betaine (LAPB), and combinations thereof.

The cleansing composition can comprise a amphoteric surfactant level from about 0.5 wt % to about 20 wt %, from about 1 wt % to about 15 wt %, from about 2 wt % to about 13 wt %, from about 3 wt % to about 15 wt %, and/or from about 5 wt % to about 10 wt %.

The surfactant system may have a weight ratio of anionic surfactant to amphoteric surfactant from about 0.4:1 to about 1.25:1, may have a weight ratio of anionic surfactant to amphoteric surfactant from about 0.5:1 to about 1.1:1, and may have a weight ratio of anionic surfactant to amphoteric surfactant from about 0.6:1 to about 1:1. In some examples, the ratio of anionic surfactant to amphoteric surfactant may be less than 1.1:1, and may be less than 1:1.

The surfactant system may further comprise one or more non-ionic surfactants and the non-ionic surfactant can be selected from the group consisting alkyl polyglucoside, alkyl glycoside, acyl glucamide and mixture thereof. Non-limiting examples of alkyl glucosides can include decyl glucoside, cocoyl glucoside, lauroyl glucoside and combination thereof.

Non-limiting examples of acyl glucamide can include lauroyl/myristoyl methyl glucamide, capryloyl/caproyl methyl glucamide, lauroyl/myristoyl methyl glucamide, cocoyl methyl glucamide and combinations thereof.

The composition can contain a non-ionic detersive surfactants that can include cocamide, cocamide methyl MEA, cocamide DEA, cocamide MEA, cocamide MIPA, lauramide DEA, lauramide MEA, lauramide MIPA, myristamide DEA, myristamide MEA, PEG-20 cocamide MEA, PEG-2 cocamide, PEG-3 cocamide, PEG-4 cocamide, PEG-5 cocamide, PEG-6 cocamide, PEG-7 cocamide, PEG-3 lauramide, PEG-5 lauramide, PEG-3 oleamide, PPG-2 cocamide, PPG-2 hydroxyethyl cocamide, and mixtures thereof.

### B. Cationic Polymer

A cleansing composition can include a cationic polymer to allow formation of a coacervate. As can be appreciated, the cationic charge of a cationic polymer can interact with an anionic charge of a surfactant to form the coacervate. Suitable cationic polymers can include: (a) a cationic guar polymer, (b) a cationic non-guar galactomannan polymer, (c) a cationic starch polymer, (d) a cationic copolymer of acrylamide monomers and cationic monomers, (e) a synthetic, non-crosslinked, cationic polymer, which may or may not form lyotropic liquid crystals upon combination with the detersive surfactant, and (f) a cationic cellulose polymer. In certain examples, more than one cationic polymer can be included.

A cationic polymer can be included by weight of the cleansing composition at about 0.05% to about 3%, about 0.075% to about 2.0%, or at about 0.1% to about 1.0%. Cationic polymers can have cationic charge densities of from about 0.2 meq/g to about 2.2 meq/g, from about 0.3 meq/g to about 2.0 meq/g, from about 0.4 meq/g to about 1.8 meq/g; from about 0.5 meq/g to about 1.7 meq/g and from about 0.6 meq/g to about 1.3. The charge densities can be measured at the pH of intended use of the cleansing composition. (e.g., at about pH 3 to about pH 9; or about pH 4 to about pH 8). The average molecular weight of cationic polymers can generally be between about 10,000 and 10 million, between about 50,000 and about 5 million, and between about 100,000 and about 3 million, and between about 300,000 and about 3 million and between about 100,000 and about 2.5 million. Low molecular weight cationic polymers can be used. Low molecular weight cationic polymers can have greater translucency in the liquid carrier of a cleansing composition. The cationic polymer can be a single type, such as the cationic guar polymer guar hydroxypropyltrimonium chloride having a weight average molecular weight of about 2.5 million g/mol or less, and the cleansing composition can have an additional cationic polymer of the same or different types.

### Cationic Guar Polymer

The cationic polymer can be a cationic guar polymer, which is a cationically substituted galactomannan (guar) gum derivative. Suitable guar gums for guar gum derivatives can be obtained as a naturally occurring material from the seeds of the guar plant. As can be appreciated, the guar molecule is a straight chain mannan which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums can be obtained through reactions between the hydroxyl groups of the polygalactomannan and reactive quaternary ammonium compounds. The degree of substitution of the cationic groups onto the guar structure can be sufficient to provide the requisite cationic charge density described above.

A cationic guar polymer can have a weight average molecular weight (“M.Wt.”) of less than about 3 million g/mol, and can have a charge density from about 0.05 meq/g to about 2.5 meq/g. Alternatively, the cationic guar polymer can have a weight average M.Wt. of less than 1.5 million g/mol, from about 150 thousand g/mol to about 1.5 million g/mol, from about 200 thousand g/mol to about 1.5 million g/mol, from about 300 thousand g/mol to about 1.5 million g/mol, and from about 700,000 thousand g/mol to about 1.5 million g/mol. The cationic guar polymer can have a charge density from about 0.2 meq/g to about 2.2 meq/g, from about 0.3 meq/g to about 2.0 meq/g, from about 0.4 meq/g to about 1.8 meq/g; from about 0.5 meq/g to about 1.7 meq/g and from about 0.6 meq/g to about 1.3.

A cationic guar polymer can have a weight average M.Wt. of less than about 1 million g/mol, and can have a charge density from about 0.1 meq/g to about 2.5 meq/g. A cationic guar polymer can have a weight average M.Wt. of less than 900 thousand g/mol, from about 150 thousand to about 800 thousand g/mol, from about 200 thousand g/mol to about 700 thousand g/mol, from about 300 thousand to about 700 thousand g/mol, from about 400 thousand to about 600 thousand g/mol, from about 150 thousand g/mol to about 800 thousand g/mol, from about 200 thousand g/mol to about 700 thousand g/mol, from about 300 thousand g/mol to about 700 thousand g/mol, and from about 400 thousand g/mol to about 600 thousand g/mol. A cationic guar polymer has a charge density from about 0.2 meq/g to about 2.2 meq/g, from about 0.3 meq/g to about 2.0 meq/g, from about 0.4 meq/g to about 1.8 meq/g; and from about 0.5 meq/g to about 1.5 meq/g.

A cleansing composition can include from about 0.01% to less than about 0.7%, by weight of the cleansing composition of a cationic guar polymer, from about 0.04% to about 0.55%, by weight, from about 0.08% to about 0.5%, by weight, from about 0.16% to about 0.5%, by weight, from about 0.2% to about 0.5%, by weight, from about 0.3% to about 0.5%, by weight, and from about 0.4% to about 0.5%, by weight.

The cationic guar polymer can be formed from quaternary ammonium compounds which conform to general Formula II:

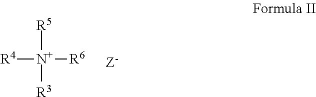

wherein where R³, R⁴ and R⁵ are methyl or ethyl groups; and R⁶ is either an epoxyalkyl group of the general Formula III:

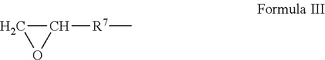

or R⁶ is a halohydrin group of the general Formula IV:

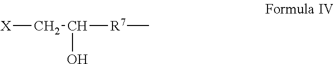

wherein R⁷ is a C₁ to C₃ alkylene; X is chlorine or bromine, and Z is an anion such as Cl−, Br−, I− or HSO₄−.

Suitable cationic guar polymers can conform to the general formula V:

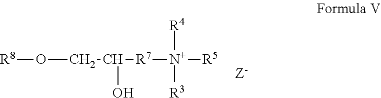

wherein R⁸ is guar gum; and wherein R⁴, R⁵, R⁶ and R⁷ are as defined above; and wherein Z is a halogen. Suitable cationic guar polymers can conform to Formula VI:

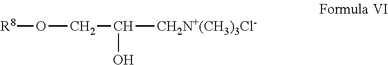

wherein R⁸ is guar gum.

Suitable cationic guar polymers can also include cationic guar gum derivatives, such as guar hydroxypropyltrimonium chloride. Suitable examples of guar hydroxypropyltrimonium chlorides can include the Jaguar® series commercially available from Solvay S.A., Hi-Care Series from Rhodia, and N-Hance and AquaCat from Ashland Inc. Jaguar® C-500 has a charge density of 0.8 meq/g and a M.Wt. of 500,000 g/mole; Jaguar Optima has a cationic charge density of about 1.25 meg/g and a M.Wt. of about 500,000 g/moles; Jaguar® C-17 has a cationic charge density of about 0.6 meq/g and a M.Wt. of about 2.2 million g/mol; Jaguar® and a cationic charge density of about 0.8 meq/g; Hi-Care 1000 has a charge density of about 0.7 meq/g and a M.Wt. of about 600,000 g/mole; N-Hance 3269 and N-Hance 3270, have a charge density of about 0.7 meq/g and a M.Wt. of about 425,000 g/mole; N-Hance 3196 has a charge density of about 0.8 meq/g and a M.Wt. of about 1,100,000 g/mole; and AquaCat CG518 has a charge density of about 0.9 meq/g and a M.Wt. of about 50,000 g/mole. N-Hance BF-13 and N-Hance BF-17 are borate (boron) free guar polymers. N-Hance BF-13 has a charge density of about 1.1 meq/g and M.W.t of about 800,000 and N-Hance BF-17 has a charge density of about 1.7 meq/g and M.W.t of about 800,000. BF-17 has a charge density of about 1.7 meq/g and M.W.t of about 800,000. BF-17 has a charge density of about 1.7 meq/g and M.W.t of about 800,000. BF-17 has a charge density of about 1.7 meq/g and M.W.t of about 800,000. BF-17 has a charge density of about 1.7 meq/g and M.W.t of about 800,000.

### Cationic Non-Guar Galactomannan Polymer

The cationic polymer can be a galactomannan polymer derivative. Suitable galactomannan polymer can have a mannose to galactose ratio of greater than 2:1 on a monomer to monomer basis and can be a cationic galactomannan polymer derivative or an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers can be present in the endosperm of seeds of the Leguminosae family Galactomannan polymers are made up of a combination of mannose monomers and galactose monomers. The galactomannan molecule is a straight chain mannan branched at regular intervals with single membered galactose units on specific mannose units. The mannose units are linked to each other by means of β(1-4) glycosidic linkages. The galactose branching arises by way of an α(1-6) linkage. The ratio of mannose monomers to galactose monomers varies according to the species of the plant and can be affected by climate. Non Guar Galactomannan polymer derivatives can have a ratio of mannose to galactose of greater than 2:1 on a monomer to monomer basis. Suitable ratios of mannose to galactose can also be greater than 3:1 or greater than 4:1. Analysis of mannose to galactose ratios is well known in the art and is typically based on the measurement of the galactose content.

The gum for use in preparing the non-guar galactomannan polymer derivatives can be obtained from naturally occurring materials such as seeds or beans from plants. Examples of various non-guar galactomannan polymers include Tara gum (3 parts mannose/1 part galactose), Locust bean or Carob (4 parts mannose/1 part galactose), and Cassia gum (5 parts mannose/1 part galactose).

A non-guar galactomannan polymer derivative can have a M. Wt. from about 1,000 g/mol to about 10,000,000 g/mol, and a M.Wt. from about 5,000 g/mol to about 3,000,000 g/mol.

The cleansing compositions described herein can include galactomannan polymer derivatives which have a cationic charge density from about 0.5 meq/g to about 7 meq/g. The galactomannan polymer derivatives can have a cationic charge density from about 1 meq/g to about 5 meq/g. The degree of substitution of the cationic groups onto the galactomannan structure can be sufficient to provide the requisite cationic charge density.

A galactomannan polymer derivative can be a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general Formulas II to VI, as defined above.

Cationic non-guar galactomannan polymer derivatives formed from the reagents described above can be represented by the general Formula VII:

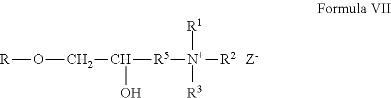

wherein R is the gum. The cationic galactomannan derivative can be a gum hydroxypropyltrimethylammonium chloride, which can be more specifically represented by the general Formula VIII:

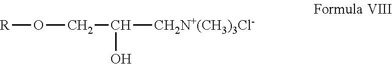

The galactomannan polymer derivative can be an amphoteric galactomannan polymer derivative having a net positive charge, obtained when the cationic galactomannan polymer derivative further comprises an anionic group.

A cationic non-guar galactomannan can have a ratio of mannose to galactose which is greater than about 4:1, a M.Wt. of about 100,000 g/mol to about 500,000 g/mol, a M.Wt. of about 50,000 g/mol to about 400,000 g/mol, and a cationic charge density from about 1 meq/g to about 5 meq/g, and from about 2 meq/ g to about 4 meq/g.

Cleansing compositions can include at least about 0.05% of a galactomannan polymer derivative by weight of the composition. The cleansing compositions can include from about 0.05% to about 2%, by weight of the composition, of a galactomannan polymer derivative.

### Cationic Starch Polymers

Suitable cationic polymers can also be water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The cleansing compositions described herein can include cationically modified starch polymers at a range of about 0.01% to about 10%, and/or from about 0.05% to about 5%, by weight of the composition.

The cationically modified starch polymers disclosed herein have a percent of bound nitrogen of from about 0.5% to about 4%.

The cationically modified starch polymers can have a molecular weight from about 850,000 g/mol to about 15,000,000 g/mol and from about 900,000 g/mol to about 5,000,000 g/mol.

Cationically modified starch polymers can have a charge density of from about 0.2 meq/g to about 5 meq/g, and from about 0.2 meq/g to about 2 meq/g. The chemical modification to obtain such a charge density can include the addition of amino and/or ammonium groups into the starch molecules. Non-limiting examples of such ammonium groups can include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. Further details are described in Solarek, D. B., Cationic Starches in Modified Starches: Properties and Uses, Wurzburg, O. B., Ed., CRC Press, Inc., Boca Raton, Fla. 1986, pp 113-125 which is hereby incorporated by reference. The cationic groups can be added to the starch prior to degradation to a smaller molecular weight or the cationic groups may be added after such modification.

A cationically modified starch polymer can have a degree of substitution of a cationic group from about 0.2 to about 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution can be determined using proton nuclear magnetic resonance spectroscopy (“¹H NMR”) methods well known in the art. Suitable ¹H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be selected from a variety of sources such as tubers, legumes, cereal, and grains. For example, starch sources can include corn starch, wheat starch, rice starch, waxy corn starch, oat starch, cassaya starch, waxy barley, waxy rice starch, glutenous rice starch, sweet rice starch, amioca, potato starch, tapioca starch, oat starch, sago starch, sweet rice, or mixtures thereof. Suitable cationically modified starch polymers can be selected from degraded cationic maize starch, cationic tapioca, cationic potato starch, and mixtures thereof. Cationically modified starch polymers are cationic corn starch and cationic tapioca.

The starch, prior to degradation or after modification to a smaller molecular weight, can include one or more additional modifications. For example, these modifications may include cross-linking, stabilization reactions, phosphorylations, and hydrolyzations. Stabilization reactions can include alkylation and esterification.

Cationically modified starch polymers can be included in a cleansing composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

The starch can be readily soluble in water and can form a substantially translucent solution in water. The transparency of the composition is measured by Ultra-Violet/Visible (“UV/VIS”) spectrophotometry, which determines the absorption or transmission of UV/VIS light by a sample, using a Gretag Macbeth Colorimeter Color. A light wavelength of 600 nm has been shown to be adequate for characterizing the degree of clarity of cleansing compositions.

### Cationic Copolymer of an Acrylamide Monomer and a Cationic Monomer

A cleansing composition can include a cationic copolymer of an acrylamide monomer and a cationic monomer, wherein the copolymer has a charge density of from about 1.0 meq/g to about 3.0 meq/g. The cationic copolymer can be a synthetic cationic copolymer of acrylamide monomers and cationic monomers.

Suitable cationic polymers can include:
(i) an acrylamide monomer of the following Formula IX:

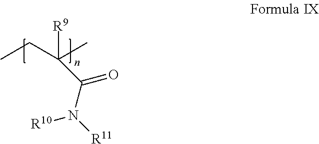

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula X:

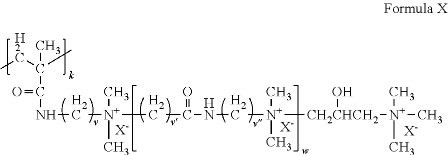

where k=1, each of v, v′, and v″ is independently an integer of from 1 to 6, w is zero or an integer of from 1 to 10, and X⁻ is an anion.

A cationic monomer can conform to Formula X where k=1, v=3 and w=0, z=1 and X⁻ is Cl⁻ to form the following structure (Formula XI):

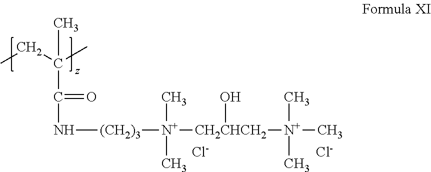

As can be appreciated, the above structure can be referred to as diquat.

A cationic monomer can conform to Formula X wherein v and v″ are each 3, v′=1, w=1, y=1 and X⁻ is Cl⁻, to form the following structure of Formula XII:

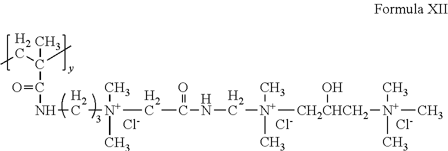

The structure of Formula XII can be referred to as triquat.

The acrylamide monomer can be either acrylamide or methacrylamide.

The cationic copolymer can be AM:TRIQUAT which is a copolymer of acrylamide and 1,3 -Propanediaminium,N-[2-[[[dimethyl[3-[(2-methyl-1-oxo-2-propenyl)amino]propyl]ammonio]acetyl]amino]ethyl]2-hydroxy-N,N,N′,N′,N′- pentamethyl,trichloride. AM:TRIQUAT is also known as polyquaternium 76 (PQ76). AM:TRIQUAT can have a charge density of 1.6 meq/g and a M.Wt. of 1.1 million g/mol.

The cationic copolymer can include an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

The cationic copolymer can include a cationic monomer selected from the group consisting of: trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

The cationic copolymer can be formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters can be cationized esters of the (meth)acrylic acid containing a quaternized N atom. Cationized esters of the (meth)acrylic acid containing a quaternized N atom can be quaternized dialkylaminoalkyl (meth)acrylates with C₁ to C₃ in the alkyl and alkylene groups. The cationized esters of the (meth)acrylic acid containing a quaternized N atom can be selected from the group consisting of ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. The cationized esters of the (meth)acrylic acid containing a quaternized N atom can be dimethylaminoethyl acrylate, which is quaternized with an alkyl halide, or with methyl chloride or benzyl chloride or dimethyl sulfate (ADAME-Quat). The cationic monomer when based on (meth)acrylamides are quaternized dialkylaminoalkyl(meth)acrylamides with C₁ to C₃ in the alkyl and alkylene groups, or dimethylaminopropylacrylamide, which is quaternized with an alkyl halide, or methyl chloride or benzyl chloride or dimethyl sulfate.

The cationic monomer based on a (meth)acrylamide can be a quaternized dialkylaminoalkyl(meth)acrylamide with C₁ to C₃ in the alkyl and alkylene groups. The cationic monomer based on a (meth)acrylamide can be dimethylaminopropylacrylamide, which is quaternized with an alkyl halide, especially methyl chloride or benzyl chloride or dimethyl sulfate.

The cationic monomer can be a hydrolysis-stable cationic monomer. Hydrolysis-stable cationic monomers can be, in addition to a dialkylaminoalkyl(meth)acrylamide, any monomer that can be regarded as stable to the OECD hydrolysis test. The cationic monomer can be hydrolysis-stable and the hydrolysis-stable cationic monomer can be selected from the group consisting of: diallyldimethylammonium chloride and water-soluble, cationic styrene derivatives.

The cationic copolymer can be a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). The cationic copolymer can be formed from acrylamide and acrylamidopropyltrimethylammonium chloride, wherein the acrylamidopropyltrimethylammonium chloride has a charge density of from about 1.0 meq/g to about 3.0 meq/g.

The cationic copolymer can have a charge density of from about 1.1 meq/g to about 2.5 meq/g, from about 1.1 meq/g to about 2.3 meq/g, from about 1.2 meq/g to about 2.2 meq/g, from about 1.2 meq/g to about 2.1 meq/g, from about 1.3 meq/g to about 2.0 meq/g, and from about 1.3 meq/g to about 1.9 meq/g.

The cationic copolymer can have a M.Wt. from about 100 thousand g/mol to about 2 million g/mol, from about 300 thousand g/mol to about 1.8 million g/mol, from about 500 thousand g/mol to about 1.6 million g/mol, from about 700 thousand g/mol to about 1.4 million g/mol, and from about 900 thousand g/mol to about 1.2 million g/mol.

The cationic copolymer can be a trimethylammoniopropylmethacrylamide chloride-N-Acrylamide copolymer, which is also known as AM:MAPTAC. AM:MAPTAC can have a charge density of about 1.3 meq/g and a M.Wt. of about 1.1 million g/mol. The cationic copolymer can be AM:ATPAC. AM:ATPAC can have a charge density of about 1.8 meq/g and a M.Wt. of about 1.1 million g/mol.

### Synthetic Polymers

A cationic polymer can be a synthetic polymer that is formed from:

i) one or more cationic monomer units, and optionally
ii) one or more monomer units bearing a negative charge, and/or
iii) a nonionic monomer,

wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers

The cationic polymers can be water soluble or dispersible, non-crosslinked, and synthetic cationic polymers which have the structure of Formula XIII:

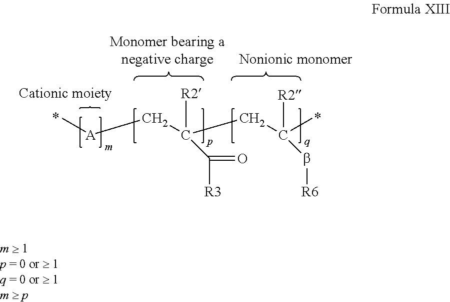

where A, may be one or more of the following cationic moieties:

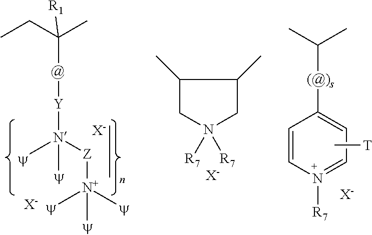

=amido, alkylamido, ester, ether, alkyl or alkylaryl;

where Y=C1-C22 alkyl, alkoxy, alkylidene, alkyl or aryloxy;
where ψ=C1-C22 alkyl, alkyloxy, alkyl aryl or alkyl arylox;.
where Z=C1-C22 alkyl, alkyloxy, aryl or aryloxy;
where R1=H, C1-C4 linear or branched alkyl;
where s=0 or 1, n=0 or ≥1;
where T and R7=C1-C22 alkyl; and
where X−=halogen, hydroxide, alkoxide, sulfate or alkylsulfate.

Where the monomer bearing a negative charge is defined by R2′=H, C₁-C₄ linear or branched alkyl and R3 is:

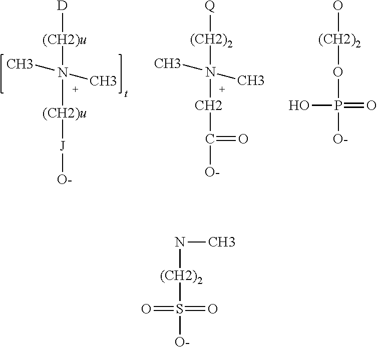

where D=O, N, or S;
where Q=NH₂ or O;
where u=1-6;
where t=0-1; and
where J=oxygenated functional group containing the following elements P, S, C.

Where the nonionic monomer is defined by R2″=H, C₁-C₄ linear or branched alkyl, R6=linear or branched alkyl, alkyl aryl, aryl oxy, alkyloxy, alkylaryl oxy and β is defined as

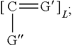

and
where G′ and G″ are, independently of one another, O, S or N—H and L=0 or 1.

Suitable monomers can include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of suitable cationic monomers can include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers can include quaternary monomers of formula —NR₃⁺, wherein each R can be identical or different, and can be a hydrogen atom, an alkyl group comprising 1 to 10 carbon atoms, or a benzyl group, optionally carrying a hydroxyl group, and including an anion (counter-ion). Examples of suitable anions include halides such as chlorides, bromides, sulphates, hydrosulphates, alkylsulphates (for example comprising 1 to 6 carbon atoms), phosphates, citrates, formates, and acetates.

Suitable cationic monomers can also include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride. Additional suitable cationic monomers can include trimethyl ammonium propyl (meth)acrylamido chloride.

Examples of monomers bearing a negative charge include alpha ethylenically unsaturated monomers including a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge can include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers can include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers can also include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X⁻) in association with the synthetic cationic polymers can be any known counterion so long as the polymers remain soluble or dispersible in water, in the cleansing composition, or in a coacervate phase of the cleansing composition, and so long as the counterions are physically and chemically compatible with the essential components of the cleansing composition or do not otherwise unduly impair product performance, stability or aesthetics. Non limiting examples of suitable counterions can include halides (e.g., chlorine, fluorine, bromine, iodine), sulfate, and methylsulfate.

The cationic polymer described herein can also aid in repairing damaged hair, particularly chemically treated hair by providing a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair. The hydrophobic layer can return the hair to a generally virgin-like, healthier state. Lyotropic liquid crystals are formed by combining the synthetic cationic polymers described herein with the aforementioned anionic detersive surfactant component of the cleansing composition. The synthetic cationic polymer has a relatively high charge density. It should be noted that some synthetic polymers having a relatively high cationic charge density do not form lyotropic liquid crystals, primarily due to their abnormal linear charge densities. Such synthetic cationic polymers are described in PCT Patent App. No. WO 94/06403 which is incorporated by reference. The synthetic polymers described herein can be formulated in a stable cleansing composition that provides improved conditioning performance, with respect to damaged hair.

Cationic synthetic polymers that can form lyotropic liquid crystals have a cationic charge density of from about 2 meq/gm to about 7 meq/gm, and/or from about 3 meq/gm to about 7 meq/gm, and/or from about 4 meq/gm to about 7 meq/gm. The cationic charge density is about 6.2 meq/gm. The polymers also have a M. Wt. of from about 1,000 to about 5,000,000, and/or from about 10,000 to about 2,000,000, and/or from about 100,000 to about 2,000,000.

Cationic synthetic polymers that provide enhanced conditioning and deposition of benefit agents but do not necessarily form lytropic liquid crystals can have a cationic charge density of from about 0.7 meq/gm to about 7 meq/gm, and/or from about 0.8 meq/gm to about 5 meq/gm, and/or from about 1.0 meq/gm to about 3 meq/gm. The polymers also have a M.Wt. of from about 1,000 g/mol to about 5,000,000 g/mol, from about 10,000 g/mol to about 2,000,000 g/mol, and from about 100,000 g/mol to about 2,000,000 g/mol.

### Cationic Cellulose Polymer

Suitable cationic polymers can be cellulose polymers. Cationic cellulose polymers can have cationic charge densities of from about 0.2 meq/g to about 2.2 meq/g, from about 0.3 meq/g to about 2.0 meq/g, from about 0.4 meq/g to about 1.8 meq/g; from about 0.5 meq/g to about 1.7 meq/g and from about 0.6 meq/g to about 1.3. Suitable cellulose polymers can include salts of hydroxyethyl cellulose reacted with trimethyl ammonium substituted epoxide, referred to in the industry (CTFA) as Polyquaternium 10 and available from Dow/Amerchol Corp. (Edison, N.J., USA) in their Polymer LR, JR, and KG series of polymers. Other suitable types of cationic cellulose can include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose can include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

Additional cationic polymers are also described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which is incorporated herein by reference.

Techniques for analysis of formation of complex coacervates are known in the art. For example, microscopic analyses of the compositions, at any chosen stage of dilution, can be utilized to identify whether a coacervate phase has formed. Such coacervate phase can be identifiable as an additional emulsified phase in the composition. The use of dyes can aid in distinguishing the coacervate phase from other insoluble phases dispersed in the composition. Additional details about the use of cationic polymers and coacervates are disclosed in U.S. Pat. No. 9,272,164 which is incorporated by reference.

### C. Hydroxamic Acids and Hydroxamic Acid Derivatives

A hydroxamic acid is a class of organic compounds bearing the functional group RC(O)N(OH)R′, with R and R′ as organic residues and CO as a carbonyl group.

Hydroxamic acid derivative of the present invention refers to a class of organic compounds bearing the functional group RC(O)N(O)R′, with R and R′ as organic residues. The hydroxamic acid derivative may be a salt of hydroxamic acid. The hydroxamic acid derivative may be an olamine salt of the hydroxamic acid.

The antimicrobial active in accordance with the invention is at least one of hydroxamic acids or hydroxamic acid derivatives. The hydroxamic acid may be piroctone, caprylhydroxamic acid, or benzohydroxamic acid. The hydroxamic acid may be caprylhydroxamic acid. It is preferred that the hydroxamic acid derivative is piroctone olamine. Therefore, the antimicrobial active according to present invention may be at least one of piroctone, caprylhydroxamic acid, benzohydroxamic acid or piroctone olamine. The antimicrobial active according to present invention may be at least one of caprylhydroxamic acid or piroctone olamine. It is most preferred that the antimicrobial active is piroctone olamine.

Piroctone is a cyclic hydroxamic acid that consists of 1-hydroxypyridin-2-one bearing methyl and 2,4,4-trimethylpentyl substituents at positions 4 and 6 respectively. The CAS number is 50650-76-5 and the compound has the general formula (a) as below:

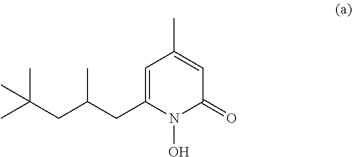

Caprylhydroxamic acid is an amino acid derived from coconut oil. It is a preservative and broad spectrum anti-fungal agent. The CAS number is 7377-03-9 and the compound has the general formula (b) as below:

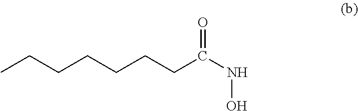

Benzohydroxamic acid is one of hydroxamic acids. The CAS number is 495-18-1 and the compound has the general formula (c) as below general formula (c) as below:

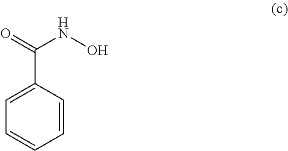

Piroctone Olamine is an olamine salt of the hydroxamic acid derivative piroctone which is a typical antimicrobial active. It is commonly known as piroctone ethanolamine with the trade name Octopirox®.

The piroctone olamine according to the present invention is a 1:1 compound of 1- hydroxy-4-methyl-6-(2,4,4-trimethylpentyl)-2(7/-/)-pyridinone with 2-aminoethanol and is also designated 1-hydroxy-4-methyl-6-(2,4,4-trimethylpentyl)-2(7/-/) pyridinone monoethanolamine salt. The CAS number is 68890-66-4 and the compound has the general formula (d) as below:

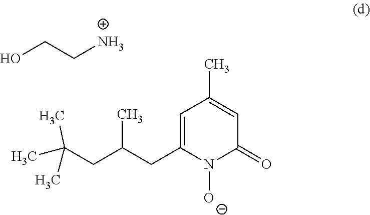

Amount of the antimicrobial active which is at least one of hydroxamic acids or hydroxamic acid derivatives in the composition of the invention would depend on the type of the topical composition and the precise nature of other antimicrobial actives used. The present invention may comprise 0.01 to 10 wt % of said antimicrobial active; may comprise 0.1 to 5 wt %; may comprise 0.5 to 3 wt % by weight of the composition.

### D. Liquid Carrier

As can be appreciated, cleansing compositions can desirably be in the form of pourable liquid under ambient conditions. Inclusion of an appropriate quantity of a liquid carrier can facilitate the formation of a cleansing composition having an appropriate viscosity and rheology. A cleansing composition can include, by weight of the composition, about 20% to about 95%, by weight, of a liquid carrier, and about 60% to about 85%, by weight, of a liquid carrier. The liquid carrier can be an aqueous carrier such as water.

### E. Optional Components

As can be appreciated, cleansing compositions described herein can include a variety of optional components to tailor the properties and characteristics of the composition. As can be appreciated, suitable optional components are well known and can generally include any components which are physically and chemically compatible with the essential components of the cleansing compositions described herein. Optional components should not otherwise unduly impair product stability, aesthetics, or performance. Individual concentrations of optional components can generally range from about 0.001% to about 10%, by weight of a cleansing composition. Optional components can be further limited to components which will not impair the clarity of a translucent cleansing composition.

Suitable optional components which can be included in a cleansing composition can include co-surfactants, deposition aids, conditioning agents (including hydrocarbon oils, fatty esters, silicones), anti-dandruff agents, suspending agents, viscosity modifiers, dyes, nonvolatile solvents or diluents (water soluble and insoluble), pearlescent aids, foam boosters, pediculocides, pH adjusting agents, perfumes, preservatives, chelants, proteins, skin active agents, sunscreens, UV absorbers, and vitamins The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of non-limiting materials that can be added to the composition herein.

### Conditioning Agents

A cleansing composition can include a silicone conditioning agent. Suitable silicone conditioning agents can include volatile silicone, non-volatile silicone, or combinations thereof. If including a silicone conditioning agent, the agent can be included from about 0.01% to about 10%, by weight of the composition, from about 0.1% to about 8%, from about 0.1% to about 5%, and/or from about 0.2% to about 3%. Examples of suitable silicone conditioning agents, and optional suspending agents for the silicone, are described in U.S. Reissue Pat. No. 34,584, U.S. Pat Nos. 5,104,646, and 5,106,609, each of which is incorporated by reference herein. Suitable silicone conditioning agents can have a viscosity, as measured at 25° C., from about 20 centistokes (“csk”) to about 2,000,000 csk, from about 1,000 csk to about 1,800,000 csk, from about 50,000 csk to about 1,500,000 csk, and from about 100,000 csk to about 1,500,000 csk.

The dispersed silicone conditioning agent particles can have a volume average particle diameter ranging from about 0.01 micrometer to about 50 micrometer. For small particle application to hair, the volume average particle diameters can range from about 0.01 micrometer to about 4 micrometer, from about 0.01 micrometer to about 2 micrometer, from about 0.01 micrometer to about 0.5 micrometer. For larger particle application to hair, the volume average particle diameters typically range from about 5 micrometer to about 125 micrometer, from about 10 micrometer to about 90 micrometer, from about 15 micrometer to about 70 micrometer, and/or from about 20 micrometer to about 50 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), which is incorporated herein by reference.

Silicone emulsions suitable for the cleansing compositions described herein can include emulsions of insoluble polysiloxanes prepared in accordance with the descriptions provided in U.S. Pat. No. 4,476,282 and U.S. Patent Application Publication No. 2007/0276087 each of which is incorporated herein by reference. Suitable insoluble polysiloxanes include polysiloxanes such as alpha, omega hydroxy-terminated polysiloxanes or alpha, omega alkoxy-terminated polysiloxanes having a molecular weight within the range from about 50,000 to about 500,000 g/mol. The insoluble polysiloxane can have an average molecular weight within the range from about 50,000 to about 500,000 g/mol. For example, the insoluble polysiloxane may have an average molecular weight within the range from about 60,000 to about 400,000; from about 75,000 to about 300,000; from about 100,000 to about 200,000; or the average molecular weight may be about 150,000 g/mol. The insoluble polysiloxane can have an average particle size within the range from about 30 nm to about 10 micron. The average particle size may be within the range from about 40 nm to about 5 micron, from about 50nm to about 1 micron, from about 75 nm to about 500 nm, or about 100 nm, for example.

Other classes of silicones suitable for the cleansing compositions described herein can include i) silicone fluids, including silicone oils, which are flowable materials having viscosity less than about 1,000,000 csk as measured at 25° C.; ii) aminosilicones, which contain at least one primary, secondary or tertiary amine; iii) cationic silicones, which contain at least one quaternary ammonium functional group; iv) silicone gums; which include materials having viscosity greater or equal to 1,000,000 csk as measured at 25° C.; v) silicone resins, which include highly cross-linked polymeric siloxane systems; vi) high refractive index silicones, having refractive index of at least 1.46, and vii) mixtures thereof.

Alternatively, the cleansing composition can be substantially free of silicones. As used herein, substantially free of silicones means from about 0 to about 0.2 wt. %.

### Organic Conditioning Materials

The conditioning agent of the cleansing compositions described herein can also include at least one organic conditioning material such as oil or wax, either alone or in combination with other conditioning agents, such as the silicones described above. The organic material can be non-polymeric, oligomeric or polymeric. The organic material can be in the form of an oil or wax and can be added in the cleansing formulation neat or in a pre-emulsified form. Suitable examples of organic conditioning materials can include: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

### Emulsifiers

A variety of anionic and nonionic emulsifiers can be used in the cleansing composition of the present invention. The anionic and nonionic emulsifiers can be either monomeric or polymeric in nature. Monomeric examples include, by way of illustrating and not limitation, alkyl ethoxylates, alkyl sulfates, soaps, and fatty esters and their derivatives. Polymeric examples include, by way of illustrating and not limitation, polyacrylates, polyethylene glycols, and block copolymers and their derivatives. Naturally occurring emulsifiers such as lanolins, lecithin and lignin and their derivatives are also non-limiting examples of useful emulsifiers.

### Chelating Agents

The cleansing composition can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term include alkali metal, alkaline earth, ammonium, substituted ammonium (i.e. monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. No. 5,747,440. U.S. Pat. Nos. 5,284,972 and 5,747,440 are each incorporated by reference herein. Suitable chelants can further include histidine.

Levels of an EDDS chelant or histidine chelant in the cleansing compositions can be low. For example, an EDDS chelant or histidine chelant can be included at about 0.01%, by weight. Above about 10% by weight, formulation and/or human safety concerns can arise. The level of an EDDS chelant or histidine chelant can be at least about 0.01%, by weight, at least about 0.05%, by weight, at least about 0.1%, by weight, at least about 0.25%, by weight, at least about 0.5%, by weight, at least about 1%, by weight, or at least about 2%, by weight, by weight of the cleansing composition.

### Gel Network

A cleansing composition can also include a fatty alcohol gel network. Gel networks are formed by combining fatty alcohols and surfactants in the ratio of from about 1:1 to about 40:1, from about 2:1 to about 20:1, and/or from about 3:1 to about 10:1. The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. Gel networks can provide a number of benefits to cleansing compositions. For example, a gel network can provide a stabilizing benefit to cosmetic creams and hair conditioners. In addition, gel networks can provide conditioned feel benefits to hair conditioners and shampoos.

A fatty alcohol can be included in the gel network at a level by weight of from about 0.05%, by weight, to about 14%, by weight. For example, the fatty alcohol can be included in an amount ranging from about 1%, by weight, to about 10%, by weight, and/or from about 6%, by weight, to about 8%, by weight.

Suitable fatty alcohols include those having from about 10 to about 40 carbon atoms, from about 12 to about 22 carbon atoms, from about 16 to about 22 carbon atoms, and/or about 16 to about 18 carbon atoms. These fatty alcohols can be straight or branched chain alcohols and can be saturated or unsaturated. Nonlimiting examples of fatty alcohols include cetyl alcohol, stearyl alcohol, behenyl alcohol, and mixtures thereof. Mixtures of cetyl and stearyl alcohol in a ratio of from about 20:80 to about 80:20 are suitable.

A gel network can be prepared by charging a vessel with water. The water can then be heated to about 74° C. Cetyl alcohol, stearyl alcohol, and surfactant can then be added to the heated water. After incorporation, the resulting mixture can passed through a heat exchanger where the mixture is cooled to about 35° C. Upon cooling, the fatty alcohols and surfactant crystallized can form crystalline gel network. Table 1 provides the components and their respective amounts for an example gel network composition.

To prepare the gel network pre-mix of Table 1, water is heated to about 74° C. and the fatty alcohol and gel network surfactant are added to it in the quantities depicted in Table 1. After incorporation, this mixture is passed through a mill and heat exchanger where it is cooled to about 32° C. As a result of this cooling step, the fatty alcohol, the gel network surfactant, and the water form a crystalline gel network.

TABLE 1

| Premix | % |
|---|---|
| Gel Network Surfactant¹ | 11.00 |
| Stearyl Alcohol | 8% |
| Cetyl Alcohol | 4% |
| Water | QS |

¹For anionic gel networks, suitable gel network surfactants above include surfactants with a net negative charge including sulfonates, carboxylates and phosphates among others and mixtures thereof.

For cationic gel networks, suitable gel network surfactants above include surfactants with a net positive charge including quaternary ammonium surfactants and mixtures thereof.
For Amphoteric or Zwitterionic gel networks, suitable gel network surfactants above include surfactants with both a positive and negative charge at product usage pH including betaines, amine oxides, sultaines, amino acids among others and mixtures thereof.

### Benefit Agents

A cleansing composition can further include one or more benefit agents. Exemplary benefit agents include, but are not limited to, particles, colorants, perfume microcapsules, gel networks, and other insoluble skin or hair conditioning agents such as skin silicones, natural oils such as sun flower oil or castor oil. The benefit agent can be selected from the group consisting of: particles; colorants; perfume microcapsules; gel networks; other insoluble skin or hair conditioning agents such as skin silicones, natural oils such as sun flower oil or castor oil; and mixtures thereof.

### Suspending Agent

A cleansing composition can include a suspending agent at concentrations effective for suspending water-insoluble material in dispersed form in the compositions or for modifying the viscosity of the composition. Such concentrations range from about 0.05% to about 10%, and from about 0.3% to about 5.0%, by weight of the compositions. As can be appreciated however, suspending agents may not be necessary when certain glyceride ester crystals are included as certain glyceride ester crystals can act as suitable suspending or structuring agents.

Suitable suspending agents can include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Other suitable suspending agents can include crystalline suspending agents which can be categorized as acyl derivatives, long chain amine oxides, and mixtures thereof. Examples of such suspending agents are described in U.S. Pat. No. 4,741,855, which is incorporated herein by reference. Suitable suspending agents include ethylene glycol esters of fatty acids having from 16 to 22 carbon atoms. The suspending agent can be an ethylene glycol stearates, both mono and distearate, but particularly the distearate containing less than about 7% of the mono stearate. Other suitable suspending agents include alkanol amides of fatty acids, having from about 16 to about 22 carbon atoms, alternatively from about 16 to about 18 carbon atoms, suitable examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters as previously described. Long chain acyl derivatives, ethylene glycol esters of long chain carboxylic acids, long chain amine oxides, and alkanol amides of long chain carboxylic acids can also be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C₁₆, C₁₈ and tallow amido benzoic acid species of this family, which are commercially available from Stepan Company (Northfield, Ill., USA).

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine Still other suitable suspending agents include di(hydrogenated tallow)phthalic acid amide, and crosslinked maleic anhydride-methyl vinyl ether copolymer.

Other suitable suspending agents include crystallizable glyceride esters. For example, in certain embodiments, suitable glyceride esters are hydrogenated castor oils such as trihydroxystearin or dihydroxystearin. Examples of additional crystallizable glyceride esters can include the substantially pure triglyceride of 12-hydroxystearic acid. 12-hydroxystearic acid is the pure form of a fully hydrogenated triglyceride of 12-hydrox-9-cis-octadecenoic acid. As can be appreciated, many additional glyceride esters are possible. For example, variations in the hydrogenation process and natural variations in castor oil can enable the production of additional suitable glyceride esters from castor oil.

### Viscosity Modifiers

Viscosity modifiers can be used to modify the rheology of a cleansing composition. Suitable viscosity modifiers can include Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF67, all supplied by Hercules, ethylene oxide and/or propylene oxide based polymers with tradenames CARBOWAX PEGs, POLYOX WASRs, and UCON FLUIDS, all supplied by Amerchol. Sodium chloride can also be used as a viscosity modifier. Other suitable rheology modifiers can include cross-linked acrylates, cross-linked maleic anhydride co-methylvinylethers, hydrophobically modified associative polymers, and mixtures thereof.

The cleaning composition may have a viscosity of greater than about 2000 cP. The cleansing composition may have a viscosity of about 2000 cP to about 20,000 cP; may have a viscosity of from about 2500 cps to about 15,000 cps; may have a viscosity of from about 3000 cP to about 12,000 cP; may have a viscosity of from about 3500 cP to about 11,000 cP; may have a viscosity of from about 2,000 cP to about 9,000 cP; as measured at 26.7° C., as measured by the Cone/Plate Viscosity Measurement Test Method, described herein.

### Dispersed Particles

Dispersed particles as known in the art can be included in a cleansing composition. If including such dispersed particles, the particles can be incorporated, by weight of the composition, at levels of about 0.025% or more, about 0.05% or more, about 0.1% or more, about 0.25% or more, and about 0.5% or more. However, the cleansing compositions can also contain, by weight of the composition, about 20% or fewer dispersed particles, about 10% or fewer dispersed particles, about 5% or fewer dispersed particles, about 3% or fewer dispersed particles, and about 2% or fewer dispersed particles.

As can be appreciated, a cleansing composition can include still further optional components. For example, amino acids can be included. Suitable amino acids can include water soluble vitamins such as vitamins B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

Anti-dandruff agents can be included. As can be appreciated, the formation of a coacervate can facilitate deposition of the anti-dandruff agent to the scalp.

A cleansing composition can optionally include pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names. The compositions can also include antimicrobial agents which are useful as cosmetic biocides and antidandruff agents including: water soluble components such as piroctone olamine, water insoluble components such as 3,4,4′- trichlorocarbanilide (trichlosan), triclocarban and zinc pyrithione.

One or more stabilizers can be included. For example, one or more of ethylene glycol distearate, citric, citrate, a preservative such as kathon, sodium benzoate, sodium salicylate and ethylenediaminetetraacetic acid (“EDTA”) can be included to improve the lifespan of a cleansing composition.

### Product Form

The hair care compositions of the present invention may be presented in typical hair care formulations. They may be in the form of solutions, dispersion, emulsions, powders, talcs, encapsulated, spheres, spongers, solid dosage forms, foams, and other delivery mechanisms. The compositions the present invention may be hair tonics, leave-on hair products such as treatment, and styling products, rinse-off hair products such as shampoos and personal cleansing products, and treatment products; and any other form that may be applied to hair.

### Method of Making a Cleansing Composition

A cleansing composition described herein can be formed similarly to known cleansing compositions. For example, the process of making a cleansing composition can include the step of mixing the surfactant, cationic polymer, piroctone olamine and liquid carrier together to form a cleansing composition.

### Test Methods

Determination of Wt % Sodium Chloride in Composition

### 1. Argentometry Method to Measure Wt % Inorganic Chloride Salts

The weight % inorganic chloride salt in formula can be measured using a potentiometric method where the chloride ions in the composition are titrated with silver nitrate. The silver ions react with the chloride ions from the composition to form an insoluble precipitate, silver chloride. The method uses an electrode (Mettler Toledeo DM141) that is designed for potentiometric titrations of anions that precipitate with silver. The largest change in the signal occurs at the equivalence point when the amount of added silver ions is equal to the amount of chloride ions in solution. The concentration of silver nitrate solution used should be calibrated using a sodium chloride solution containing a standard and known amount of sodium chloride to confirm that the results match the known concentration. This type of titration involving a silver ion is known as argentometry and is commonly used to determine the amount of chloride present in a sample.

### Methods to Determine Lack of In Situ Coacervate in Composition Prior to Dilution

### 1. Microscopy Method to Determine Lack of In Situ Coacervate

The composition does not contain in situ coacervate. Lack of in situ coacervate can be determined using a microscope. The composition is mixed to homogenize, if needed. Then, the composition is sampled onto a microscope slide and mounted on a microscope, per typical microscopy practices. The sample is viewed at, for example, a 10× or 20× objective. If in situ coacervate is present in the sample, an amorphous, gel-like phase with about 20 nm to about 200 nm particle size can be seen throughout the sample. This amorphous, gel-like phases can be described as gel chunks or globs. The composition containing surfactants substantially free from sulfates, cationic deposition polymers, a low level of inorganic salt and a hydroxamic acid or or hydroxamic acid derivative will not have amorphous, gel-like phases when viewed under a microscope.

### 2. Clarity by % Transmittance Method to Determine Lack of In Situ Coacervate

The composition does not contain in situ coacervate. Lack of in situ coacervate can also be determined by composition clarity. A composition that does not contain in situ coacervate will be clear, if it does not contain any ingredients that would otherwise give it a hazy appearance. Composition clarity can be measure by % Transmittance. For this assessment to determine if the composition lacks coacervate, the composition should be made without silicones, opacifiers, non-silicone oils, micas, gums or anionic rheology modifiers and other ingredients that would cause the shampoo to have a hazy appearance. Another material that may cause a hazy appearance are glycol diesters. The level of glycol diesters in a composition may need to be reduced in order to achieve a % Transmittance (%T) of greater than 70%. It is believed that adding these ingredients would not cause in situ coacervate to form prior to use, however these ingredients will obscure measurement of clarity by % Transmittance. Clarity can be measured by % Transmittance (%T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the %T is measured by the spectrophotometer at 600 nm. Alternatively, multiple samples can be measured simultaneously by using a spectrophotometer such as the SpectraMax M-5 available from Molecular Devices. Multiple samples are transferred into a 96 well visible flat bottom plate (Greiner part #655-001), ensuring that no air bubbles are within the samples. The flat bottom plate is placed within the SpectraMax M-5 and %T measured using the Software Pro v.STM software available from Molecular Devices. A composition containing surfactants substantially free from sulfates, cationic deposition polymers and a low level of inorganic salt will not have amorphous, gel-like phases when viewed under a microscope. The composition containing surfactants substantially free from sulfates, cationic deposition polymers, a low level of inorganic salt and a hydroxamic acid or hydroxamic acid derivative will have a percent transparency (%T) of at least about 70% transmittance at 600 nm.

### 3. Clarity by Visual Assessment Method to Determine Lack of In Situ Coacervate

The composition does not contain in situ coacervate. Lack of in situ coacervate can be determined by composition clarity. A composition that does not contain in situ coacervate will be clear. Composition clarity can also be determined by visual assessment. For this assessment, the composition should be made without silicones, opacifiers, non-silicone oils, micas, gums or anionic rheology modifiers and other ingredients that would cause the shampoo to have a hazy appearance. It is believed that adding these ingredients would not cause in situ coacervate to form prior to use, however these ingredients will obscure measurement of clarity by visual assessment. For this assessment, the composition is made and immediately sampled into a clear, glass jar of at least 1 inch width. The cap is screwed on the jar, finger-tight. The jar is stored at ambient temperatures (20-25° C.), away from direct sunlight, until there are no air bubbles in the sample. The sample may contain no air bubbles in as soon as 1 day or up to 7 days. Then the sample is visually inspected to determine if it is clear or hazy. If the sample is visually clear, then there is no in situ coacervate. The composition containing surfactants substantially free from sulfates, cationic deposition polymers, a low level of inorganic salt and a hydroxamic acid or hydroxamic acid derivative will be clear when assessed visually be this method.

### 4. Lasentec FBRM Method to Determine Lack of In Situ Coacervate

The composition does not contain in situ coacervate. Lack of in situ coacervate can also be measured using Lasentec FBRM Method with no dilution. A Lasentec Focused Beam Reflectance Method (FBRM) [model S400A available from Mettler Toledo Corp] may be used to determine floc size and amount as measured by chord length and particle counts/sec (counts per sec). The composition containing surfactants substantially free from sulfates, cationic deposition polymers, a low level of inorganic salt and a hydroxamic acid or hydroxamic acid derivative does not contain flocs. The composition with other materials added does not contain flocs of different particle size than the particle size of the other materials added.

### 5. In Situ Coacervate Centrifuge Method to Determine Lack of In Situ Coacervate

The composition does not contain in situ coacervate. Lack of in situ coacervate can also be measured by centrifuging a composition and measuring in situ coacervate gravimetrically. For this method, the composition should be made without a suspending agent to allow for separation of an in situ coacervate phase. The composition is centrifuged for 20 minutes at 9200 rpm using a Beckman Couller TJ25 centrifuge. Several time/rpm combinations can be used. The supernatant is then removed and the remaining settled in situ coacervate assessed gravimetrically. % In Situ Coacervate is calculated as the weight of settled in situ coacervate as a percentage of the weight of composition added to the centrifuge tube using the equation below. This quantifies the percentage of the composition that participates in the in situ coacervate phase. The % In Situ Coacervate for the composition containing surfactants substantially free from sulfates, cationic deposition polymers, a low level of inorganic salt and a hydroxamic acid or hydroxamic acid derivative is 0%.

$$\% In Situ Coacervate = \frac{Weight of settled in situ coacervate}{Weight of composition added to centrifuge tube} \times 100$$

### 6. Visual Assessment of Phase Separation to Determine Lack of In Situ Coacervate

The composition does not contain in situ coacervate. Lack of in situ coacervate can also be measured be determined by visual assessment of phase separation. For this method, the composition should be made without a suspending agent to allow for separation of an in situ coacervate phase. The composition is made and immediately sampled into a glass jar. An example jar is a 20 ml scintillation vial. The cap is screwed on the jar, finger-tight. The jar is stored at ambient temperatures (20-25° C.), away from direct sunlight. A composition containing in situ coacervate will form a separated phase on the bottom of the container. This phase will form in as short as 3 days, but could take up to 9 months depending on the viscosity of the composition. The composition containing surfactants substantially free from sulfates, cationic deposition polymers, a low level of inorganic salt and a hydroxamic acid or hydroxamic acid derivative will not form a separated phase.

### Measures of Improved Performance Due to NO In Situ Coacervate Prior to Dilution

The composition does not contain in situ coacervate prior to dilution. Because of this, coacervate quantity and quality upon dilution is better than a composition that does contain in situ coacervate prior to dilution. This provides better wet conditioning and deposition of actives from a composition that does not contain coacervate prior to dilution compared to a composition that does contain coacervate prior to dilution.

### 1. Measurement of % Transmittance (%T) During Dilution

Techniques for analysis of formation of complex coacervates are known in the art. One method to assess coacervate formation upon dilution for a transparent or translucent composition is to use a spectrophotometer to measure the percentage of light transmitted through the diluted sample (%T). As percent light transmittance (%T) values measured of the dilution decrease, typically higher levels of coacervate are formed. Dilutions samples at various weight ratios of water to composition can be prepared, for example 2 parts of water to 1 part composition (2:1), or 7.5 parts of water to 1 part composition (7.5:1), or 16 parts of water to 1 part composition (16:1), or 34 parts of water to 1 part composition (34:1), and the %T measured for each dilution ratio sample. Examples of possible dilution ratios may include 2:1, 3:1, 5:1, 7.5:1, 11:1, 16:1, 24:1, or 34:1. By averaging the %T values for samples that span a range of dilution ratios, it is possible to simulate and ascertain how much coacervate a composition on average would form as a consumer applies the composition to wet hair, lathers, and then rinses it out. Average %T can be calculated by taking the numerical average of individual %T measurements for the following dilution ratios: 2:1, 3:1, 5:1, 7.5:1, 11:1, 16:1, 24:1, and 34:1. Lower average %T indicates more coacervate is formed on average as a consumer applies the composition to wet hair, lathers and then rinses it out. The composition containing surfactants substantially free from sulfates, cationic deposition polymers, a low level of inorganic salt and a hydroxamic acid or hydroxamic acid derivative will have a lower average %T than a similar composition with a higher level of inorganic salt.

T can be measured using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to insure no air bubbles are within the sample before the %T is measured by the spectrophotometer at 600 nm. Alternatively, multiple samples can be measured simultaneously by using a spectrophotometer such as the SpectraMax M-5 available from Molecular Devices. Multiple dilution samples can be prepared within a 96 well plate (VWR catalog #82006-448) and then transferred to a 96 well visible flat bottom plate (Greiner part #655-001), ensuring that no air bubbles are within the sample. The flat bottom plate is placed within the SpectraMax M-5 and %T measured using the Software Pro v.STM software available from Molecular Devices.

### 2. Assessment of Coacervate Floc Size upon Dilution

Coacervate floc size upon dilution can be assessed visually. Dilutions samples at various weight ratios of water to composition can be prepared, for example 2 parts of water to 1 part composition (2:1), or 7.5 parts of water to 1 part composition (7.5:1), or 16 parts of water to 1 part composition (16:1), or 34 parts of water to 1 part composition (34:1), and the %T measured for each dilution ratio sample. Examples of possible dilution ratios may include 2:1, 3:1, 5:1, 7.5:1, 11:1, 16:1, 24:1, or 34:1. The composition containing surfactants substantially free from sulfates, cationic deposition polymers, a low level of inorganic salt and a hydroxamic acid or hydroxamic acid derivative will have larger coacervate flocs than a similar composition with a higher level of inorganic salt. Larger coacervate flocs can indicate a better quality coacervate that provides better wet conditioning and deposition of actives.

### 3. Wet Combing Force Method

Hair switches of 4 grams general population hair at 8 inches length are used for the measurement. Each hair switch is treated with 4 cycles (1 lather/rinse steps per cycle, 0.1 gm cleansing composition/gm hair on each lather/rinse step, drying between each cycle) with the cleansing composition. Four switches are treated with each shampoo. The hair is not dried after the last treatment cycle. While the hair is wet, the hair is pulled through the fine tooth half of two Beautician 3000 combs. Force to pull the hair switch through the combs is measured by a friction analyzer (such as Instron or MTS tensile measurement) with a load cell and outputted in gram-force (gf). The pull is repeated for a total of five pulls per switch. Average wet combing force is calculated by averaging the force measurement from the five pulls across the four hair switches treated with each cleansing composition. Data can be shown as average wet combing force through one or both of the two combs. The composition containing surfactants substantially free from sulfates, cationic deposition polymers, a low level of inorganic salt and a hydroxamic acid or hydroxamic acid derivative will have a lower combing force than a similar composition with a higher level of inorganic salt.

### 4. Deposition Method

Deposition of actives can be measured in vitro on hair tresses or in vivo on panelist's heads. The composition is dosed on a hair tress or panelist head at a controlled amount and washed according to a conventional washing protocol. For a hair tress, the tress can be sampled and tested by an appropriate analytical measure to determine quantity deposited of a given active. To measure deposition on a panelist's scalp, the hair is then parted on an area of the scalp to allow an open-ended glass cylinder to be held on the surface while an aliquot of an extraction solution is added and agitated prior to recovery and analytical determination of a given active. To measure deposition on a panelist's hair, a given amount of hair is sampled and then tested by an appropriate analytical measure to determine quantity deposited of a given active. The composition containing surfactants substantially free from sulfates, cationic deposition polymers, a low level of inorganic salt and a hydroxamic acid or hydroxamic acid derivative will have higher deposition than a similar composition with a higher level of inorganic salt.

### Measurement of Anti-Dandruff Agent Deposition

Anti-dandruff agent deposition, for example a hydroxamic acid or or hydroxamic acid derivative such as piroctone olamine, deposition in-vivo on scalp can be determined by ethanol extraction of the agent after the scalp has been treated with a surfactant-soluble agent containing cleansing composition and rinsed off. The concentration of agent in the extraction solvent or solution is measured by HPLC. Quantitation is made by reference to a standard curve. The concentration detected by HPLC is converted into an amount collected in grams by using the concentration multiplied by volume.

The percent agent deposited can be calculated using the following equation:

$$\% agent deposited = \frac{\frac{grams of agent deposited}{area of scalp extracted}}{\frac{( wt . \% agent in shampoo ) \times ( grams of shampoo applied )}{area of scalp treated}} \times 100 \%$$

### Viscosity Measures

### A. Viscosity Measure

The viscosities of the examples are measured by a Cone/Plate Controlled Stress Brookfield Rheometer R/S Plus, by Brookfield Engineering Laboratories, Stoughton, Mass. The cone used (Spindle C-75-1) has a diameter of 75 mm and 1° angle. The liquid viscosity is determined using a steady state flow experiment at constant shear rate of 2 s⁻¹ and at temperature of 26.7° C. The sample size is about 2.5 ml to about 3 ml and the total measurement reading time is 3 minutes. Initial Viscosity may be measured immediately after making Initial Viscosity may also be measured after confirming that there are no air bubbles in the sample. The sample is stored at ambient temperatures (20-25° C.), away from direct sunlight, until there are no air bubbles in the sample. The sample may contain no air bubbles in as soon as 1 day or up to 7 days.

### B. Measure of Consistent Viscosity over Aging

Compositions that achieve acceptable viscosity at a higher pH will have more consistent viscosity over aging. Compositions containing a hydroxamic acid or hydroxamic acid derivative will achieve acceptable viscosity at higher pH than similar compositions that do not contain a hydroxamic acid or hydroxamic acid derivative.

Elevated temperature is a common method which may be used to accelerate aging and is a common technique used in the industry. For example, 65° C. or 40° C. can be used to accelerate aging. A sample of the composition is placed at the elevated temperature for a time period. Time at 65° C. can be 1 week, 2 weeks or 3 weeks. Time at 40° C. can be 1 months, 2 months, 3 months, 4 months, 5 months or 6 months. After the time period at the elevated temperature, the sample is pulled and equilibrated to ambient room temperature (22° C.-27° C.). This equilibration time period may be completed as soon as 3 hours or may require up to 24 hours. Sample containers may be placed in a water bath at ambient room temperature to accelerate equilibration to ambient room temperature to about 1 hour. Then viscosity of the sample is measured using the Viscosity Measure above.

The change in viscosity between initial viscosity and viscosity after accelerated aging can be calculated various ways. One way to calculate this change is % Increase in Viscosity. There may be other ways to calculate this change.

$$\% Increase in Viscosity = \frac{Viscosity after Accelerated Aging - Initial Viscosity}{Viscosity Initial} \times 100$$

### pH Method

First, calibrate the Mettler Toledo Seven Compact pH meter. Do this by turning on the pH meter and waiting for 30 seconds. Then take the electrode out of the storage solution, rinse the electrode with distilled water, and carefully wipe the electrode with a scientific cleaning wipe, such as a Kimwipe®. Submerse the electrode in the pH 4 buffer and press the calibrate button. Wait until the pH icon stops flashing and press the calibrate button a second time. Rinse the electrode with distilled water and carefully wipe the electrode with a scientific cleaning wipe. Then submerse the electrode into the pH 7 buffer and press the calibrate button a second time. Wait until the pH icon stops flashing and press the calibrate button a third time. Rinse the electrode with distilled water and carefully wipe the electrode with a scientific cleaning wipe. Then submerse the electrode into the pH 10 buffer and press the calibrate button a third time. Wait until the pH icon stops flashing and press the measure button. Rinse the electrode with distilled water and carefully wipe with a scientific cleaning wipe. Submerse the electrode into the testing sample and press the read button. Wait until the pH icon stops flashing and record the value.

### Lather Characterization

### 1. Kruss DFA100 Lather Characterization

A cleansing composition dilution of 10 parts by weight water to 1 part by weight cleanser is prepared. The shampoo dilution is dispensed into the Kruss DFA100 which generates the lather and measures lather properties.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

The following Examples illustrate various cleansing compositions. Each cleansing composition is prepared by conventional formulation and mixing techniques.

The total sodium chloride in the tables below is calculated based on the product specifications from the suppliers. Some of the surfactants used in the examples below are sourced in a liquid mixture containing the surfactant at some active concentration, water, and often sodium chloride at some level generated during synthesis of the surfactant. For example, a common surfactant synthesis that produces sodium chloride as a byproduct is the synthesis of cocamidopropyl betaine. In this synthesis, an amidoamine is reacted with sodium monochloroacetate to produce betaine and sodium chloride. This is one example of a surfactant synthesis that produces sodium chloride as a byproduct. Public supplier documents including example Certificate of Analysis and Technical Specification documents list activity by wt % or solids by wt % and wt % sodium chloride. Using these specifications and the surfactant activity in the composition, inherent levels of sodium chloride coming in with the surfactants can be summed up for a given composition and added to any sodium chloride that is directly added to the composition. While surfactants are a common raw material that introduces sodium chloride into the formula, other materials can also be checked for content of sodium chloride to include in the overall sodium chloride calculation. For calculation of total inorganic salt, this total sodium chloride is added to any other inorganic salts that are added through a raw material or intentionally.

The initial viscosity and viscosity after 1 week at 65° C. in Error! Reference source not found. and Table 4 and Table 6 is determined using the Viscosity Measure, described herein. Initial viscosity of the composition is measured immediately after making or up to 7 days after making after confirming that there are no air bubbles in the composition. A sample of the composition is placed in an oven set to 65° C. for 1 week. After 1 week at 65° C., the sample is pulled and equilibrated to ambient room temperature (22° C.-27° C.) wherein this equilibration time period may be completed as soon as 3 hours or may require up to 24 hours. Sample containers may be placed in a water bath at ambient room temperature to accelerate equilibration to ambient room temperature. Then viscosity of the sample is measured using the Viscosity Measure, described herein. The change in viscosity is calculated by % Increase in Viscosity.

$$\% Increase in Viscosity = \frac{Viscosity after Accelerated Aging - Initial Viscosity}{Initial Viscosity} \times 100$$

For Examples 1-6 and Comparative Examples 1-4, the in situ coacervate is determined as follows. The examples are prepared as described herein. The example is made and immediately put in a clear, glass jar of at least 1 inch width. The cap is screwed on the jar, finger-tight. The example is stored at ambient temperatures (20-25° C.), away from direct sunlight until there is no air bubbles left in the sample (up to 7 days depending on viscosity of the sample). Then the composition is inspected to see if either haze or precipitate is visually detectable. If either haze or precipitate is present, it is determined that the composition has in situ coacervate. If neither haze nor precipitate is present, it is determined that there is no in situ coacervate. It is believed that the shampoo product would have improved conditioning performance as compared to examples where in situ coacervate formed.

The example is inspected to determine if haze could be detected visually or by % Transmittance Method. If the example is clear, then there is no in situ coacervate and it is believed that the shampoo product will have improved conditioning performance as compared to examples where in situ coacervate formed. If haze is detected in the example, then there is in situ coacervate and it is believed that the example will be less preferred by consumers.

As used herein, “visually detect” or “visually detectable” means that a human viewer can visually discern the quality of the example with the unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 1 meter.

TABLE 2
Experiments with combination surfactants,
cationic polymers and piroctone olamine.

| | Ex. 1 | Ex. 2 | Ex. 3 | Ex. 4 |
|---|---|---|---|---|
| | (wt. %) | (wt. %) | (wt. %) | (wt. %) |
| Lauramidopropyl | 9.75 | 9.75 | 9.75 | 9.75 |
| Betaine¹ | | | | |
| Sodium Cocoyl | 6.00 | 6.00 | 6.00 | 6.00 |
| Isethionate² | | | | |
| Polyquaternium 10³ | 0.8 | — | — | — |
| Polyquaternium 10⁴ | — | 0.6 | 0.25 | 0.25 |
| Guar | — | — | — | — |
| Hydroxypropyl- | | | | |
| trimonium | | | | |
| Chloride⁵ | | | | |
| Piroctone Olamine⁶ | 0.5 | 0.5 | 0.5 | 0.8 |
| Sodium Benzoate⁷ | 0.45 | 0.45 | 0.45 | 0.45 |
| Sodium Salicylate⁸ | 0.45 | 0.45 | 0.45 | 0.45 |
| Citric Acid⁹ | To pH | To pH | To pH | To pH |
| | 5.6 | 5.5 | 5.5 | 5.5 |
| Added Sodium | 0 | 0 | 0 | 0 |
| Chloride¹⁰ | | | | |
| Water and Perfume | Q.S. to | Q. S. to | Q.S. to | Q.S. to |
| | 100 | 100 | 100 | 100 |
| Total Sodium | 0.07 | 0.07 | 0.07 | 0.07 |
| Chloride (including | | | | |
| from surfactant) | | | | |
| Viscosity as made | 8689 cP | 6958 cP | 2188 cP | 4076 cP |
| Viscosity after | 10156 cP | 10295 cP | 3913 cP | 6991 cP |
| 1 week at 65° C. | | | | |
| % Increase in | 17% | 48% | 79% | 72% |
| Viscosity after | | | | |
| 1 week at 65° C. | | | | |
| Contains in situ | No | No | No | No |
| coacervate prior | | | | |
| to dilution? | | | | |
| % T of Composition | 84 | 95 | 89 | 78 |

Examples 1-4 contain 0.07% total sodium chloride and 0.5-0.8% Piroctone Olamine. No in situ coacervate prior to dilution is observed. Examples 1-4 have initial viscosity greater than 2000 cP, which is determined to be sufficient, and would be acceptable to consumers. Examples 1-4 increase viscosity over 1 week at 65° C. less than 80%, which is determined to be acceptable to consumers. While increase in viscosity less than 80% is acceptable, the consumer preference will continue to improve as the viscosity increase is reduced. It is anticipated that Examples 1-4 will have good product performance as made and over time and will be consumer preferred.

TABLE 3
Comparative Examples

| | C1 | C2 |
|---|---|---|
| Lauramidopropyl Betaine¹ | 9.75 | 9.75 |
| Sodium Cocoyl Isethionate² | 6.00 | 6.00 |
| Polyquaternium 10³ | 0.8 | |
| Polyquaternium 10⁴ | | 0.6 |
| Guar Hydroxypropyltrimonium | | |
| Chloride⁵ | | |
| Piroctone Olamine⁶ | 0.5 | 0.5 |
| Sodium Benzoate⁷ | 0.45 | 0.45 |
| Sodium Salicylate⁸ | 0.45 | 0.45 |
| Citric Acid⁹ | To pH | To pH |
| | 5.5-6.0 | 5.5-6.0 |
| Added Sodium Chloride¹⁰ | 1.25 | 1.40 |
| Water, Perfume and Optional | Q.S. | Q.S. |
| Components | to 100 | to 100 |
| Total Sodium Chloride (including | 1.32 | 1.47 |
| from surfactant) | | |
| Contains in situ coacervate prior | Yes | Yes |
| to dilution? | | |
| % T of Composition | 5.9 | 2.5 |

Comparative Examples 1 (C1) and 2 (C2) are hazy with %T of 5.9 and %T of 2.5 respectively, indicating the presence of in situ coacervate. C1 and C2 are believed to have less conditioning performance and will not be consumer preferred. As shown in C1 and C2, sulfate-free surfactant systems containing more than about 1% inorganic salt may not form compositions that are consumer preferred.

TABLE 4

| | Ex. 5 | | Ex. 6 | |
|---|---|---|---|---|
| | (wt. %) | C3 | (wt. %) | C4 |
| Lauramidopropyl Betaine¹ | 9.75 | 9.75 | 9.75 | 9.75 |
| Sodium Cocoyl Isethionate² | 6.00 | 6.00 | 6.00 | 6.00 |
| Polyquaternium 10³ | 0.6 | 0.6 | — | — |
| Polyquaternium 10⁴ | — | — | 0.4 | 0.4 |
| Guar Hydroxypropyltrimonium | — | — | — | — |
| Chloride⁵ | | | | |
| Piroctone Olamine⁶ | 0.5 | — | 0.5 | — |
| Sodium Benzoate⁷ | 0.45 | 0.45 | 0.45 | 0.45 |
| Sodium Salicylate⁸ | 0.45 | 0.45 | 0.45 | 0.45 |
| Perfume | 1 | 1 | 1 | 1 |
| Citric Acid⁹ | To pH | To pH | To pH | To pH |
| | 5.9 | 5.4 | 5.7 | 5.4 |
| Added Sodium Chloride¹⁰ | 0 | 0 | 0 | 0 |
| Water and Optional Components | Q.S. | Q.S. | Q.S. | Q.S. |
| | to 100 | to 100 | to 100 | to 100 |
| Total Sodium Chloride | 0.07 | 0.07 | 0.07 | 0.07 |
| (including from surfactant) | | | | |
| Viscosity as made | 4154 cP | 2951 cP | 2842 cP | 1591 cP |
| Viscosity after 1 week | 5710 cP | 4225 cP | 4018 cP | 2423 cP |
| at 65° C. | | | | |
| % Increase in Viscosity | 37% | 43% | 41% | 52% |
| after 1 week at 65° C. | | | | |
| Contains in situ coacervate | No | No | No | No |
| prior to dilution? | | | | |
| % T of Composition | 95 | 82 | 92 | 96 |

The impact of piroctone olamine is tested in compositions of equivalent composition aside from the addition of piroctone olamine. Surfactant types and level, cationic polymer type and level, and perfume type and level is consistent. As compared to Example 5, Comparative Example 3 does not contain piroctone olamine. As compared to Example 6, Comparative Example 4 does not contain piroctone olamine. When making these compositions, pH of the composition is decreased using Citric Acid in order to increase viscosity.

As compared to Example 5, Comparative Example 3 (C3) does not contain piroctone olamine. pH of C3 is decreased lower than pH of Example 5 to increase viscosity. However, initial viscosity of C3 is lower than initial viscosity of Example 5 even with decreasing pH of C3 lower than pH of Example 5. Because pH of C3 is lower than pH of Example 5, more surfactant hydrolysis occurs in C3 than in Example 5. As a result of this hydrolysis, C3 has a 43% Increase in Viscosity after 1 week at 65° C. whereas Example 5 has a 37% Increase in Viscosity after 1 week at 65° C. Example 5 containing piroctone olamine has a higher initial viscosity and a more consistent aged viscosity, which is anticipated to be more preferred by consumers than Comparative Example 3 (C3) which does not contain piroctone olamine and has a lower initial viscosity and less consistent aged viscosity. Also as result of more hydrolysis in C3, it is anticipated to have less consistent performance and less consumer preferred.

As compared to Example 6, Comparative Example 4 (C4) does not contain piroctone olamine. pH of C4 is decreased lower than pH of Example 6 to increase viscosity. However, initial viscosity of C4 is lower than initial viscosity of Example 6 even with decreasing pH of C4 lower than pH of Example 8. Because pH of C4 is lower than pH of Example 6, more surfactant hydrolysis occurs in C4 than in Example 6. As a result of this hydrolysis, C4 has a 52% Increase in Viscosity after 1 week at 65° C. whereas Example 6 has a 41% Increase in Viscosity after 1 week at 65° C. Example 6 containing piroctone olamine has a higher initial viscosity and a more consistent aged viscosity, which is anticipated to be more preferred by consumers than Comparative Example 4 (C4) which does not contain piroctone olamine and has a lower initial viscosity and less consistent aged viscosity. Also as result of more hydrolysis in C4, it is anticipated to have less consistent performance and less consumer preferred.

It is anticipated that the pH of Comparative Example 3 (C3) and Comparative Example 4 (C4) would need to be further decreased for initial viscosity to be equivalent to Example 5 and Example 6 respectively. Because surfactant hydrolysis is accelerated with decreasing pH, it is anticipated that the % Increase in Viscosity would be higher than current Comparative Example 3 (C3) and Comparative Example 4 (C4). This less consistent viscosity over aging is less preferred by consumers. This is demonstrated in Table 6.

TABLE 5

| | Ex. 7 | Ex. 8 | Ex. 9 | Ex. 10 | Ex. 11 | Ex. 12 |
|---|---|---|---|---|---|---|
| | (wt. %) | (wt. %) | (wt. %) | (wt. %) | (wt. %) | (wt. %) |
| Lauramidopropyl Betaine ¹ | — | 9.75 | 9.75 | 9.75 | 9.75 | 9.75 |
| Low Salt Cocamidopropyl | 9.75 | — | — | — | — | — |
| Betaine ¹¹ | | | | | | |
| Sodium Cocoyl Isethionate ² | 6.00 | 6.00 | 6.00 | 6.00 | 6.00 | 6.00 |
| Sodium Lauroyl Sarcosinate ¹² | — | 4 | 2.5 | — | — | — |
| Polyquaternium 10 ³ | 0.8 | 0.8 | 0.8 | 0.8 | 0.8 | 0.8 |
| Polyquaternium 10 ⁴ | — | — | — | — | — | — |
| Guar | — | — | — | — | — | — |
| Hydroxypropyltrimonium | | | | | | |
| Chloride ⁵ | | | | | | |
| Piroctone Olamine ⁶ | 0.5 | 0.5 | 0.5 | 0.5 | 0.5 | 0.5 |
| Sodium Benzoate ⁷ | 0.45 | 0.45 | 0.45 | 0.75 | — | 0.45 |
| Sodium Salicylate ⁸ | 0.45 | 0.45 | 0.45 | 0.45 | 0.45 | — |
| Citric Acid ⁹ | To pH 5.5-6.0 | To pH 5.5-6.0 | To pH 5.5-6.0 | To pH 5.5-6.0 | To pH 5.5-6.0 | To pH 5.5-6.0 |
| Added Sodium Chloride ¹⁰ | 0 | 0 | 0 | 0 | 0 | 0 |
| Water, Perfume and | Q.S.to 100 | Q.S. to 100 | Q.S. to 100 | Q.S. to 100 | Q.S. to 100 | Q.S. to 100 |
| Optional Components | | | | | | |
| Total Sodium Chloride | 0.06 | 0.07 | 0.07 | 0.07 | 0.07 | 0.07 |
| (including from surfactant) | | | | | | |

TABLE 6

| | Ex 13 | |
|---|---|---|
| | (wt. %) | C5 |
| Lauramidopropyl Betaine¹ | 9.75 | 9.75 |
| Sodium Cocoyl Isethionate² | 6.00 | 6.00 |
| Polyquaternium 10³ | 0.6 | 0.6 |
| Polyquaternium 10⁴ | — | — |
| Guar Hydroxypropyltrimonium | — | — |
| Chloride⁵ | | |
| Piroctone Olamine⁶ | 0.5 | — |
| Sodium Benzoate⁷ | 0.45 | 0.45 |
| Sodium Salicylate⁸ | 0.45 | 0.45 |
| Perfume | 1 | 1 |
| Citric Acid⁹ | To pH | To pH |
| | 5.9 | 5.1 |
| Added Sodium Chloride¹⁰ | 0 | 0 |
| Water and Optional Components | Q.S. to | Q.S. to |
| | 100 | 100 |
| Total Sodium Chloride (including | 0.07 | 0.07 |
| from surfactant) | | |
| Initial Viscosity | 5532 cP | 5119 cP |
| Viscosity after 1 week | 7997 cP | 8321 cP |
| at 65° C. | | |
| % Increase in Viscosity | 45% | 63% |
| after 1 week at 65° C. | | |
| Contains in situ coacervate | No | No |
| prior to dilution? | | |

### Suppliers for Examples

1. Mackam DAB-ULS available from Solvay. Specification Range: Solids=34-36%, Sodium Chloride=0-0.5%. Average values are used for calculations: Actives=35%, Sodium Chloride=0.25%.

2. Hostapon SCI-85 C available from Clariant

3. UCARE Polymer LR-30M available from Dow

4. UCARE Polymer JR-30M available from Dow

5. N-Hance 3196 Cationic Guar available from Ashland

6. Octopirox available from Clariant

7. Sodium Benzoate available from Kalama Chemical

8. Sodium Salicylate available from JQC (Huayin) Pharmaceutical Co., Ltd

9. Citric Acid USP Anhydrous Fine Granular available from Archer Daniels Midland Company

10. Sodium Chloride available from Norton International Inc.

11. Dehyton PK 45 from BASF with Sodium Chloride removed, resulting in 33.05% Dry Residue, 0.21% Sodium Chloride

12. SP Crodasinic LS30/NP MBAL available from Croda

### Combinations

A. A clear cleansing composition comprising:
from about 3 wt % to about 35 wt % of an anionic surfactant;
from about 5 wt % to about 15% of an amphoteric surfactant;
from about 0.01 wt % to about 2 wt % of a cationic polymer;
from about 0 wt % to about 1.0 wt % of inorganic salts;
from about 0.01% to about 10% of a hydroxamic acid or hydroxamic acid derivative; an aqueous carrier,
wherein the composition is substantially free of sulfate based surfactant and wherein the composition has a % T value of greater than about 70.

B. A cleaning composition according to Paragraph A, wherein the anionic surfactant is selected from the group consisting of sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

C. A cleansing composition according to Paragraph A-B, wherein the cationic polymer has a weight average molecular weight of from about 300,000 g/mol to about 3,000,000 g/mol.

D. A cleansing composition according to Paragraph A-C, wherein the cationic polymer is selected from the group consisting of cationic guars, cationic cellulose, cationic synthetic homopolymers, cationic synthetic copolymers, and combinations thereof.

E. A cleansing composition according to Paragraph A-D, wherein the cationic polymer is selected from the group consisting of hyroxypropyltrimonium guar, Polyquaternium 10, Polyquaternium 6, and combinations thereof.

F. A cleansing composition according to Paragraph A-E, wherein the charge density of the cationic polymer is from about from about 0.5 meq/g to about 1.7 meq/g.

G. A cleansing composition according to Paragraph A-F, wherein the inorganic salt is selected from the group consisting of sodium chloride, potassium chloride, sodium sulfate, ammonium chloride, sodium bromide, and combinations thereof.

H. A cleansing composition according to Paragraph A-G, wherein the hydroxamic acid or hydroxamic acid derivative is selected from the group consisting of piroctone, caprylhydroxamic acid, benzohydroxamic acid, piroctone olamine and combinations thereof.

I. A cleansing composition according to Paragraph A-H, wherein the hydroxamic acid or hydroxamic acid derivative is piroctone olamine.

J. A cleansing composition according to Paragraph A-I, wherein the composition has a viscosity greater than about 2000 cP.

K. A cleansing composition according to Paragraph A-J, wherein the composition has a viscosity of from about 2000 cP to about 20,000 cP.

L. A cleansing composition according to Paragraph A-K, wherein a ratio of the anionic surfactant to amphoteric surfactant is from about 0.4:1 to about 1.25:1.

M. A cleansing composition according to Paragraph A-L, wherein the pH is greater than about 5.5.

N. A cleansing composition according to Paragraph A-M, wherein the inorganic salt level is from about 0 wt % to about 0.9 wt %.

O. A cleansing composition according to Paragraph A-N, wherein the inorganic salt level is from about 0 wt % to about 0.8 wt %.

P. A cleansing composition according to Paragraph A-O, wherein the inorganic salt level is from about 0 wt % to about 0.2 wt %.

Q. A cleansing composition according to Paragraph A-P, wherein the amphoteric surfactant is selected from the group consisting of betaines, sultaines, hydroxysultanes, amphohydroxypropyl sulfonates, alkyl amphoactates, alkyl amphodiacetates and combination thereof.

R. A cleansing composition according to Paragraph A-Q, wherein the % T has a value of from about 70% to about 100%.

S. A cleansing composition according to Paragraph A-R, wherein the composition consists of 9 or fewer ingredients.

T. A cleansing composition according to Paragraph A-S, wherein the composition lacks in situ coacervate, as determined by the Microscopy Method to Determine Lack of In Situ Coacervate.

It will be appreciated that other modifications of the present disclosure are within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified. A level of perfume and/or preservatives may also be included in the following examples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A clear cleansing composition comprising:
from about 3 wt % to about 35 wt % of an anionic surfactant;
from about 5 wt % to about 15% of an amphoteric surfactant;
from about 0.01 wt % to about 2 wt % of a cationic polymer;
from about 0 wt % to about 1.0 wt % of inorganic salts;
from about 0.01% to about 10% of a hydroxamic acid or hydroxamic acid derivative;
an aqueous carrier,
wherein the composition is substantially free of sulfate based surfactant and wherein the composition has a % T value of greater than about 70.

2. The composition of claim 1, wherein the anionic surfactant is selected from the group consisting of sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

3. The composition of claim 1, wherein the cationic polymer has a weight average molecular weight of from about 300,000 g/mol to about 3,000,000 g/mol.

4. The composition of claim 3, wherein the cationic polymer is selected from the group consisting of cationic guars, cationic cellulose, cationic synthetic homopolymers, cationic synthetic copolymers, and combinations thereof.

5. The composition of claim 4, wherein the cationic polymer is selected from the group consisting of hyroxypropyltrimonium guar, Polyquaternium 10, Polyquaternium 6, and combinations thereof.

6. The composition of claim 1, wherein the charge density of the cationic polymer is from about from about 0.5 meq/g to about 1.7 meq/g.

7. The composition of claim 1, wherein the inorganic salt is selected from the group consisting of sodium chloride, potassium chloride, sodium sulfate, ammonium chloride, sodium bromide, and combinations thereof.

8. The composition of claim 1, wherein the hydroxamic acid or hydroxamic acid derivative is selected from the group consisting of piroctone, caprylhydroxamic acid, benzohydroxamic acid, piroctone olamine and combinations thereof.

9. The composition of claim 1, wherein the hydroxamic acid or hydroxamic acid derivative is piroctone olamine.

10. The composition of claim 1, wherein the composition has a viscosity greater than about 2000 cP.

11. The composition of claim 1, wherein the composition has a viscosity of from about 2000 cP to about 20,000 cP.

12. The composition of claim 1, wherein a ratio of the anionic surfactant to amphoteric surfactant is from about 0.4:1 to about 1.25:1.

13. The composition of claim 1, wherein the pH is greater than about 5.5.

14. The composition of claim 1, wherein the inorganic salt level is from about 0 wt % to about 0.9 wt %.

15. The composition of claim 14, wherein the inorganic salt level is from about 0 wt % to about 0.8 wt %.

16. The composition of claim 15, wherein the inorganic salt level is from about 0 wt % to about 0.2 wt %.

17. The composition of claim 1, wherein the amphoteric surfactant is selected from the group consisting of betaines, sultaines, hydroxysultanes, amphohydroxypropyl sulfonates, alkyl amphoactates, alkyl amphodiacetates and combination thereof.

18. The composition of claim 1, wherein the % T has a value of from about 70% to about 100%.

19. The composition of claim 1, wherein the composition consists of 9 or fewer ingredients.

20. The composition of claim 1, wherein the composition lacks in situ coacervate, as determined by the Microscopy Method to Determine Lack of In Situ Coacervate.
